(19) United States
(12) Patent Application Publication
(10) Pub. No.: US 2014/0288454 A1
(43) Pub. Date: Sep. 25, 2014

(54) Method For Using Exhaled Breath to Determine the Presence of Drug

(71) Applicant: Pulmonary Analytics, Lakewood, CO (US)

(72) Inventors: Frederick Mark Paz, Littleton, CO (US); David Howson, Denver, CO (US)

(73) Assignee: Pulmonary Analytics, Lakewood, CO (US)

(21) Appl. No.: 14/213,592

(22) Filed: Mar. 14, 2014

Related U.S. Application Data
(60) Provisional application No. 61/784,848, filed on Mar. 14, 2013.

(51) Int. Cl. A61B 5/00; A61B 5/097; A61B 5/087; G01N 33/497; A61B 5/091; A61B 5/08; G01N 33/00

(52) U.S. Cl. CPC A61B 5/4845; A61B 5/082; A61B 5/097; G01N 33/0047; G01N 33/497; A61B 5/091; A61B 5/087; G01N 2033/4975
USPC 600/532

(57) ABSTRACT

The present invention provides a method for determining the presence and/or the level of drug in a subject's system using exhaled breath of the subject.

### CROSS-REFERENCE TO RELATED APPLICATIONS

This application claims the priority benefit of U.S. Provisional Application No. 61/784,848, filed Mar. 14, 2013, which is incorporated herein by reference in its entirety.

### FIELD OF THE INVENTION

The present invention relates to a method for determining the presence and/or the level of drug in a subject's system using exhaled breath of the subject.

### BACKGROUND OF THE INVENTION

Exhaled breath is commonly used in sobriety (i.e., alcohol) testing. In fact, there are numerous technologies available that allow on-site sobriety testing using exhaled breath. These technologies have been used extensively with legally defensible results.

Unfortunately, testing for other illicit drugs of abuse still requires blood or urine samples, because conventional breath analysis devices are not efficient enough to be used for detecting the presence of illicit drugs in a subject. Other conventional methods for testing the presence of illicit drugs use hair, sweat or oral fluid of the subject. These non-breath testing methods are invasive and often require transporting the test subject to a hospital or other facilities for sampling by medically trained personnel. Consequently, these other non-exhaled breath testing methods result in a relatively long delay before the subject is tested for the presence of illicit drugs. At worst, this delay can lead to a false negative as the drug may have cleared the test subject's system by the time a sample is taken. At best, this delay results in a very low amount of drug presence in the test subject's system.

Therefore, there is a need for a simple on-site method that can be used to test for the presence of an illicit drug in a subject.

### SUMMARY OF THE INVENTION

Some aspects of the invention provide a method for determining the presence and/or the level of drug in a subject's system. Typically, the method includes collecting aerosol particles from exhaled breath of the subject while measuring the total volume of exhaled breath. By analyzing the collected aerosol particles or analytes, one can determine the presence or absence of drug in the subject's system. In addition, by normalizing the amount of analyte based on the volume of exhaled breath collected, one can also determine the level of drug present in the subject.

### BRIEF DESCRIPTION OF THE DRAWINGS

FIG. 1 is an isometric view of an electrostatic breath aerosol analyte collector according to this invention;

FIG. 2 is a top plan view of the electrostatic breath aerosol analyte collector of FIG. 1;

FIG. 3 is a side elevation view of the electrostatic breath aerosol analyte collector of FIG. 1;

FIG. 4 is a cross-section view of the electrostatic breath analyte collector of FIGS. 1-3 taken along section plane 4-4 in FIG. 3 and illustrating an inhalation operational mode;

FIG. 5 is a cross-section view similar to FIG. 4, but illustrating an exhalation operation mode;

FIG. 6 is an enlarged cross-section view of the extractor assembly of FIGS. 4 and 5;

FIG. 7 is a cross-section view similar to FIG. 6, but illustrating a continuous solvent flow variation of the extractor assembly;

FIG. 8 is an isometric view of an example mesh assembly component;

FIG. 9 is an isometric view of an example ionizer assembly;

FIG. 10 is an isometric view of an enhanced condensation analyte collector according to this invention;

FIG. 11 is a top plan view of the enhanced condensation analyte collection of FIG. 10;

FIG. 12 is a cross-section view of the enhanced condensation analyte collector of FIGS. 10 and 11 taken along the section plane 12-12 of FIG. 11 and showing the valve positions set for inhalation mode; and

FIG. 13 is a cross-section view similar to FIG. 12, but with the valve positions reversed for exhalation mode.

### DETAILED DESCRIPTION OF THE INVENTION

With legalization of recreational and medicinal use of cannabis in some parts of the U.S. along with other countries, in particular European countries, it is difficult for law enforcement agencies to determine whether a subject has a legal limit of cannabis in the subject's system while operating a vehicle. Some aspects of the invention provide a method for on-site determination of presence of a drug in a subject's system as well as a method for determining the level of drug present in the subject's system. In particular, methods of the invention utilize a highly efficient aerosol particulate collection system to collect samples from the subject's exhaled breath to determine the presence as well as the level of drug present within the subject's system.

Accordingly, some aspects of the invention provide a non-invasive, non-specimen based apparatus, system and/or method for detecting the presence or determining the level or quantitative amount of analytes. As used herein, the term “analytes” refers to drug metabolites or the drug itself which may be present in the exhaled breath of a subject. Exemplary analytes in exhaled breath of a subject that can be analyzed using methods and apparatus of the invention for detecting the presence of drug in the subject's system include, but are not limited to, delta-9-tetrahydrocannabionol, 11-hydrorxy-THC, 11-nor-9-carboxy-THC (THCCOOH), cannabinol; amphetamine, methamphetamine, amphetamine-d₅, THC, morphine, 6-acetymorphine, cocaine, benzoylecgonine, diazepam, oxazepam, buprenorphine, methylphenidate/ritalinic acid, tramadol, acetyl-alpha-methylfentanyl (N-[-1-(1-methyl-2-phenethyl)-4-piperidinyl]-N-phenylacetamide), acetylmethadol, allylprodine, Alphacetylmethadol, Alphameprodine, Alphamethadol, Alpha-methylfentanyl, Alpha-methylthiofentanyl, Benzethidine, Betacetylmethadol, Beta-hydroxyfentanyl, Beta-hydroxy-3-methylfentanyl, Betameprodine, Betamethadol, Betaprodine, Clonitazene, Dextromoramide, Diethylthiambutene, Difenoxin, Diampromide, Dimenoxadol, Dimepheptanol, Dimethylthiambutene, Dioxaphetyl butyrate, Dipipanone, Ethylmethylthiambutene, Etonitazene, Etoxeridine, Furethidine, Hydroxypethidine, Ketobemidone, Levomoramide, Levophenacylmorphan, 3-Methylfentanyl, 3-Methylthiofentanyl, Morpheridine, MPPP (1-methyl-4-phenyl-4-propionoxypiperidine), Noracymethadol, Norlevophanol, Normethadone, Norpipanone, Para-fluorofentanyl, PEPAP (1-(-2-phenethyl)-4-phenyl-4-acetoxypiperidine), Phenadoxone, Phenampromide, Phenomorphan, Phenoperidine, Piritramide, Proheptazine, Properidine, Propiram, Racemoramide, Thiofentanyl (N-phenyl-N-[1-(2-thienyl)ethyl-4-piperidinyl]-propamide), Tilidine, Trimeperidine, Acetorphine, Acetyldihydrocodeine, Benzylmorphine, Codeine methylbromide, Codeine-N-Oxide, Cyprenorphine, Desomorphine, Dihyromorphine, Drotebanol, Etorphine, Heroin, Hydromorphinol, Methyldesorphine, Methyldihydromorphine, Morphine methylbromide, Morphine methylsulfonate, Morphine-N-Oxide, Myrophine, Nicocodeine, Nicomorphine, Normophine, Pholcodine, Thebacon, Alpha-ethyltryptamine (etryptamin, Monase, AET, a-AT), 4-Bromo-2,5-dimethoxy-amphetamine (4-bromo-2,5-DMA; 4-bromo-2,5-dimethoxy-a-methylphenethylamine), 4-Bromo-2,5-dimethoxy-phenethylamine (alpha-desmethyl DOB; 2C-B, Nexus), 2,5-Demethoxy-amphetamine (2,5-dimethoxy-a-methylphenethylamine; 2,5-DMA), 2,5-Dimethoxy-4-ethyl-amphetamine (DOET), 4-Methoxyamphetamine (4-methoxy-a-methyl-phenethylamine; PMA), 5-Methoxy-3,4-methylenedioxy-amphetamine, 4-Methyl-2,5-dimethoxy-amphetamine (4-methyl-2,5-dimethoxy-methylphenethylamine; DOM, STP), 3,4-Methylenedioxy-amphetamine (MDA), 3,4-Methylenedioxy-methamphetamine (MDMA), 3,4-Methylenedioxy-N-ethylamphetamine (N-ethyl MDA, MDE, MDEA), N-hyroxy-3,4-methylenedioxy-amphetamine (N-hydroxy MDA), 3,4,5-Trimethoxy-amphetamine, Bufotenine (3-(b-Dimethylaminoethyl)-5-hydroxyindole; 3-(2-dimethylaminoethyl) 5-indolol; N,N-dimethylserotonin; 5-hydroxy-N,N-dimethyltryptamine; mappine), Diethyltryptamine (DET), Dimethyltryptamine (DMT), Ibogaine (Tabermanthe iboga; 7-Ethyl-6,6-b, 7,8,9,10,12,13-octahydro-2-methoxy-6,9-methano-5H-pyrido (1′,2′:1,2) azepino (5,4-b) indole), Lysergic acid diethylamide (LSD), Marihuana, Mescaline, Parahexyl (Synhexyl; 3-Hexyl-1-hydroxy-7,8,9,10-tetrahydro-6,6,9-trimethyl-6H-dibenzo(b,d)pyran), Peyote (all parts of the plant Lophosphora williamsii Lemaire), N-ethyl-3-piperidyl benzilate, N-methyl-3-piperidyl-benzilate, Psilocybin, Psilocyn, Tetrahydrocannabinols, Ethylamine analog of phencyclidine (PCE; cyclohexamine; N-ethyl-1-phenylcyclohexylamine), Pyrrolidine analog of phencyclidine (PCPy; PHP; 1-(1-phenylcyclohexyl)-pyrrolidine), Thiophene analog of phencyclidine (TPCP; TCP; 1-(1-(2-thienyl)-cyclohexyl)-piperidine), 1-(1-(2-Thienyl)cyclohexyl)pyrrolidine (TCPy), Gamma-hydroxybutyrate (GHB), Mecloqualone, Methaqualone, Aminorex (aminoxaphen; 2-amino-5-phenyl-2-oxazoline; 4,5-dihydro-5-phenyl-2-oxazolamine), Cathinone (norephedrone; 2-amino-1-phenyl-1-propanone; alpha-aminopropiophenone; 2-aminopropiophenone), Fenethylline, Methcathinone (ephedrine; methylcathinone; 2-(methylamino)-propiophenone; alpha-(methylamino)-propiophenone; monomethylpropion), (+/−)cis-4-methylaminorex, N-ethylamphetamine, N,N-dimethylamphetamine (N,N-alpha-trimethyl-benzeneethanamine; N,N-alpha-trimethylphenethylamine), N-(1-benzyl-4-piperidyl)-N-phenylpropanamide (benzylfentanyl), N-(1-(2-thienyl)methyl-4-piperidyl)-N-phenylpropanamide (thenylfentanyl), Raw opium, Opium extracts, Opium fluid, Powdered opium, Granulated opium, Tincture of opium, Codeine, Dihydroetorphine, Ethylmorphine, Etorphine hydrochloride, Hydrocodone, Hydromorphone, Metopon, Morphine, Oxycodone, Oxymorphone, Thebaine, Alfentanil, Alphaprodine, Anileridine, Bezitramide, Bulk dextropropoxyphene, Carfentanil, Dihydrocodeine, Fentanyl, Isomethadone, Levo-alphacetylmethadol (LAAM), Levomethorphan, Levorphanol, Metazocine, Methadone, Methadone-intermediate (4-cyano-2-demethylamino-4,4-diphenyl butane), Moramide-intermediate (2-methyl-3-morpholino-1,1-diphenylpropane-carboxylic acid), Pethidine (meperidine), Pethidine-intermediate-A (4-cyano-1-methyl-4-phenylpiperidine), Pethidine-intermediate-B (ethyl-4-phenylpiperidine-4-carboxylate), Pethidine-intermediate-C (1-methyl-4-phenylpiperidine-4-carboxylic acid), Phenazocine, Piminodine, Racemethorphan, Racemorphan, Remifentanil, Sufentanil, Amphetamine, Methamphetamine, Phenmetrazine, Methylphenidate, Amobarbital, Glutethimide, Pentobarbital, Phencyclidine (PCP), Secobarbital, Nabilone, Phenylacetone (P2P, phenyl-2-propanone, benzylmethyl ketone), 1-Phenylcyclohexylamine, and 1-Piperidinocyclohexanecarbontrile (PCC).

The apparatus, system and/or method of the invention are efficient, non-bulky, and user friendly both for operators and the subject. Moreover, methods of the invention are non-intrusive and non-invasive. Furthermore, methods of the invention can discern between various analytes.

Exhaled breath comprises gaseous materials, such as carbon dioxide, oxygen, water vapor, and others, and non-gaseous materials, such as liquid droplets, insoluble substances, and mixtures of the two. Materials in the exhaled breath that are not in the gaseous state at the opening of the mouth or nose when exhaled are considered to be aerosols for the purposes of this disclosure. Some examples of such aerosols include, but are not limited to, airborne solid particulates, such as dust and smoke, as well as liquid droplets that comprise drugs, biological materials, and other chemicals that can be subjected to analysis. The term “analytes” as discussed above, refers to any compound or molecule that can be used to determine the presence of drug in exhaled breath of a subject.

While many analytes do not evaporate at normal physiological (body) temperatures, fortunately however, such non-volatile analytes are present in condensates from or aerosol particulates of exhaled breath. Unfortunately, however, measurements of analytes in exhaled breath captured by conventional methods have shown excessively high variance from one measurement to the next and have been very inconsistent. Therefore, they are not reliable or useful for detecting the presence of or determining the level of drug in the subject's system. Some causes of such extreme variances may include: (i) Collector efficiency variations from one collection apparatus to another and even from one collection event to another with the same apparatus; (ii) The volume and flow rate of exhaled breaths may be highly variable from one person to another and even from the same person from one breath to another, thus presenting the collector apparatus with an irreproducible flow of breath material from which to collect samples; (iii) Surface condensation captures aerosol analytes only indirectly, thus previous state-of-the-art collectors may capture only non-predictable and non-verifiable portions of the aerosol analytes in the exhaled breath; and (iv) Condensation may cause very high dilution of dissolved analytes, thereby leading to large and irregular losses. More significantly, however is the lack of efficiency of aerosol particulate collection by conventional system. For example, on average air contains about one and one-half million particles per liter. Conventional condensate collectors allow about one million of these particles to pass through resulting in only about one-half million or less of particles being collected. Even the best medical filters allow, on average, more than two hundred thousand particles to pass through without being collected.

In contrast, the vortex collector and the electrostatic filter developed by the present inventors collect, on average, all but 2,500 particles and less than 10 particles, respectively. Such a high efficient analyte collection system significantly reduces the variability and error in determining the presence and/or the level of drug present in the subject's exhaled breath.

One particular example of the breath aerosol analyte collector is disclosed in the present inventors' U.S. Pat. No. 7,364,553, issued Apr. 29, 2008, which is incorporated herein by reference in its entirety. Some aspects of the invention for determining the presence and/or the level of drug in the subject's system will now be described with reference to breath aerosol analyte collector disclosed in U.S. Pat. No. 7,364,553. However, it should be appreciated that the scope of the invention is not limited to this particular breath aerosol analyte collector. In general, any breath aerosol analyte collector that has breath aerosol analyte collection efficiency of at least 90%, typically at least 95%, often at least 98%, more often at least 99%, and most often at least 99.9% can be used in methods of the invention. To determine the level of drug present in the subject's system, the breath aerosol analyte collector should also have a means for accurately measuring the volume of exhaled breath collected. The efficiency of volume measurement should be at least 90%, typically at least 95%, often at least 98%, more often at least 99%, and most often at least 99.9% accurate. It should be noted that more efficient collection system and more accurate volume measurement result in more accurate and more reliable result.

One particular aspect of the invention provides a method for determining the level of drug in a subject's system. Such a method typically includes: (i) collecting more than 95% of all aerosol particles from exhaled breath of a subject by having the subject exhale into a breath sample collecting apparatus (i.e., the breath aerosol analyte collector) and measuring the total volume of exhaled breath exhaled into the breath sample collecting apparatus; (ii) determining the amount of a drug metabolite (e.g., analyte) present in the collected aerosol particles; (iii) normalizing the amount of the drug metabolite in the collected aerosol particles based on the volume of exhaled breath; and (iv) determining the level of drug in the subject's system by using the normalized amount of the drug metabolite determined in said step (iii).

The method of invention can be used, for example, to measure the presence and/or the level of cannabis within the subject's system. Such a determination can be made by analyzing the metabolite of cannabis in the aerosol particulates of subject's exhaled breath. Some of the metabolites that can be tested for the presence of cannabis include, but are not limited to, Δ-9-tetrahydrocannabionol; 11-hydrorxy-tetrahydrocannabionol; 11-nor-9-carboxy-tetrahydrocannabionol; and Cannabinol. It should be noted that one can also analyze for the presence of one or more of drug metabolites. In fact, as the number of drug metabolites that are analyzed increases, the accuracy and reliability of the result also increase. Thus in some embodiments, the method of invention analyzes two or more, typically three or more, and often four or more drug metabolites.

Methods of the invention can also be used to detect the presence of or the level of an opiate in the subject's system. Exemplary opiates that can be tested for include, but are not limited to, Acetyl-alpha-methylfentanyl (N-[-1-(1-methyl-2-phenethyl)-4-piperidinyl]-N-phenylacetamide); Acetylmethadol; Allylprodine; Alphacetylmethadol; Alphameprodine; Alphamethadol; Alpha-methylfentanyl; Alpha-methylthiofentanyl; Benzethidine; Betacetylmethadol; Beta-hydroxyfentanyl; Beta-hydroxy-3-methylfentanyl; Betameprodine; Betamethadol; Betaprodine; Clonitazene; Dextromoramide; Diethylthiambutene; Difenoxin; Diampromide; Dimenoxadol; Dimepheptanol; Dimethylthiambutene; Dioxaphetyl butyrate; Dipipanone; Ethylmethylthiambutene; Etonitazene; Etoxeridine; Furethidine; Hydroxypethidine; Ketobemidone; Levomoramide; Levophenacylmorphan; 3-Methylfentanyl; 3-Methylthiofentanyl; Morpheridine; MPPP (1-methyl-4-phenyl-4-propionoxypiperidine); Noracymethadol; Norlevophanol; Normethadone; Norpipanone; Para-fluorofentanyl; PEPAP (1-(-2-phenethyl)-4-phenyl-4-acetoxypiperidine); Phenadoxone; Phenampromide; Phenomorphan; Phenoperidine; Piritramide; Proheptazine; Properidine; Propiram; Racemoramide; Thiofentanyl (N-phenyl-N-[1-(2-thienyl)ethyl-4-piperidinyl]-propamide); Tilidine; Trimeperidine; Acetorphine; Acetyldihydrocodeine; Benzylmorphine; Codeine methylbromide; Codeine-N-Oxide; Cyprenorphine; Desomorphine; Dihyromorphine; Drotebanol; Etorphine; Heroin; Hydromorphinol; Methyldesorphine; Methyldihydromorphine; Morphine methylbromide; Morphine methylsulfonate; Morphine-N-Oxide; Myrophine; Nicocodeine; Nicomorphine; Normophine; Pholcodine; and Thebacon. Other opiates and opiate derivatives that can be tested using methods of the invention include, but are not limited to, Raw opium; Opium extracts; Opium fluid; Powdered opium; Granulated opium; Tincture of opium; Codeine; Dihydroetorphine; Ethylmorphine; Etorphine hydrochloride; Hydrocodone; Hydromorphone; Metopon; Morphine; Oxycodone; Oxymorphone; Thebaine; Alfentanil; Alphaprodine; Anileridine; Bezitramide; Bulk dextropropoxyphene; Carfentanil; Dihydrocodeine; Fentanyl; Isomethadone; Levo-alphacetylmethadol (LAAM); Levomethorphan; Levorphanol; Metazocine; Methadone; Methadone-intermediate (4-cyano-2-demethylamino-4,4-diphenyl butane); Moramide-intermediate (2-methyl-3-morpholino-1,1-diphenylpropane-carboxylic acid); Pethidine (meperidine); Pethidine-intermediate-A (4-cyano-1-methyl-4-phenylpiperidine); Pethidine-intermediate-B (ethyl-4-phenylpiperidine-4-carboxylate); Pethidine-intermediate-C (1-methyl-4-phenylpiperidine-4-carboxylic acid); Phenazocine; Piminodine; Racemethorphan; Racemorphan; Remifentanil; and Sufentanil.

Methods of the invention can also be used to determine the presence of and/or the level of other drugs such as, but not limited to, stimulants, depressants, hallucinogenic drugs and other illicit drugs. Examples of other drugs and/or drug metabolites that can be tested using methods of the invention include, but are not limited to, Amphetamine; Methamphetamine; Amphetamine-d₅; THC; Morphine; 6-acetymorphine; Cocaine; Benzoylecgonine; Diazepam; Oxazepam; Buprenorphine; Methylphenidate/ritalinic acid; and Tramadol.

Still other drugs and/or drug metabolites that can be tested using methods of the invention include, but are not limited to, Alpha-ethyltryptamine (etryptamin, Monase, AET, a-AT); 4-Bromo-2,5-dimethoxy-amphetamine (4-bromo-2,5-DMA; 4-bromo-2,5-dimethoxy-a-methylphenethylamine); 4-Bromo-2,5-dimethoxy-phenethylamine (alpha-desmethyl DOB; 2C-B, Nexus); 2,5-Demethoxy-amphetamine (2,5-dimethoxy-a-methylphenethylamine; 2,5-DMA); 2,5-Dimethoxy-4-ethyl-amphetamine (DOET); 4-Methoxyamphetamine (4-methoxy-a-methyl-phenethylamine; PMA); 5-Methoxy-3,4-methylenedioxy-amphetamine; 4-Methyl-2,5-dimethoxy-amphetamine (4-methyl-2,5-dimethoxy-methylphenethylamine; DOM, STP); 3,4-Methylenedioxy-amphetamine (MDA); 3,4-Methylenedioxy-methamphetamine (MDMA); 3,4-Methylenedioxy-N-ethylamphetamine (N-ethyl MDA, MDE, MDEA); N-hyroxy-3,4-methylenedioxy-amphetamine (N-hydroxy MDA); 3,4,5-Trimethoxy-amphetamine; Bufotenine (3-(b-Dimethylaminoethyl)-5-hydroxyindole; 3-(2-dimethylaminoethyl) 5-indolol; N,N-dimethylserotonin; 5-hydroxy-N,N-dimethyltryptamine; mappine); Diethyltryptamine (DET); Dimethyltryptamine (DMT); Ibogaine (Tabermanthe iboga; 7-Ethyl-6,6-b, 7,8,9,10,12,13-octahydro-2-methoxy-6,9-methano-5H-pyrido (1′,2′:1,2) azepino (5,4-b) indole); Lysergic acid diethylamide (LSD); Marihuana; Mescaline; Parahexyl (Synhexyl; 3-Hexyl-1-hydroxy-7,8,9,10-tetrahydro-6,6,9-trimethyl-6H-dibenzo(b,d)pyran); Peyote (all parts of the plant Lophosphora williamsii Lemaire); N-ethyl-3-piperidyl benzilate; N-methyl-3-piperidyl-benzilate; Psilocybin; Psilocyn; Tetrahydrocannabinols; Ethylamine analog of phencyclidine (PCE; cyclohexamine; N-ethyl-1-phenylcyclohexylamine); Pyrrolidine analog of phencyclidine (PCPy; PHP; 1-(1-phenylcyclohexyl)-pyrrolidine); Thiophene analog of phencyclidine (TPCP; TCP; 1-(1-(2-thienyl)-cyclohexyl)-piperidine); 1-(1-(2-Thienyl)cyclohexyl)pyrrolidine (TCPy); Gamma-hydroxybutyrate (GHB); Mecloqualone; Methaqualone; Aminorex (aminoxaphen; 2-amino-5-phenyl-2-oxazoline; 4,5-dihydro-5-phenyl-2-oxazolamine); Cathinone (norephedrone; 2-amino-1-phenyl-1-propanone; alpha-aminopropiophenone; 2-aminopropiophenone); Fenethylline; Methcathinone (ephedrine; methylcathinone; 2-(methylamino)-propiophenone; alpha-(methylamino)-propiophenone; monomethylpropion); (+/−)cis-4-methylaminorex; N-ethylamphetamine; N,N-dimethylamphetamine (N,N-alpha-trimethyl-benzeneethanamine; N,N-alpha-trimethylphenethylamine); N-(1-benzyl-4-piperidyl)-N-phenylpropanamide (benzylfentanyl); N-(1-(2-thienyl)methyl-4-piperidyl)-N-phenylpropanamide (thenylfentanyl); Amphetamine; Methamphetamine; Phenmetrazine; Methylphenidate; Amobarbital; Glutethimide; Pentobarbital; Phencyclidine (PCP); Secobarbital; Nabilone; Phenylacetone (P2P, phenyl-2-propanone, benzylmethyl ketone); 1-Phenylcyclohexylamine; and 1-Piperidinocyclohexanecarbontrile (PCC).

It should be appreciated that once the aerosol particulates from exhaled breath of a subject is collected, one can analyze for the presence of a wide variety of drugs. Depending on the total volume of exhaled breath collected, one can analyze the collected aerosol particulates for one, two, three or more drugs. Typically, the volume of exhaled breath collected is at least 5 liters, often at least 10 liters, and more often at least 20 liters. Alternatively, the subject is instructed to exhale into the breath sample collecting apparatus (i.e., the breath aerosol analyte collector) for at least 1 minutes, typically at least 2 minutes and often at least 3 minutes.

Once the aerosol particulates are collected, the collection filter or collection target can be rinsed with a solvent. Suitable solvents include, but are not limited to, water, a saline solution, a buffer solution, organic solvent such as ether, alcohol (e.g., methanol, ethanol, etc.), etc. Typically, a known volume of solvent is used to dilute the collected aerosol particulates. This allows an accurate concentration measure of the analytes, e.g., drug metabolite(s). Generally, about 0.25 mL, typically about 0.5 mL, and often about 1 mL of solvent is used to dilute the collected aerosol particulates. However, it should be appreciated that the scope of the invention is not limited to such a volume of solvent. Accordingly, in some embodiments, said step (ii) of determining the amount of a drug metabolite present in the collected aerosol particles comprises: diluting said collected aerosol particles with a solvent to produce a sample solution; and determining the amount of drug metabolite in the sample solution.

The diluted aerosol particulates or the resulting solution is then analyzed for the presence and/or the amount of a particular analyte. Such analysis can be made by any of the analytical methods known to one skilled in the art including, but not limited to, a chromatography (e.g., HPLC, GC, GC/MS, etc.), mass spectrometer, infrared spectrometer, ultraviolet/visible (i.e., UV/VIS) spectrometer, nuclear magnetic resonance (NMR) spectrometer, and capillary electrophoresis.

Once the presence of a particular analyte is determined, one can compare the result with a control. For example, a control or a control value can be a threshold value indicating the presence of or the actual use of the drug by the subject. In some instance, such as in cannabis, a bystander (not the actual user) can be exposed to the “second-hand” smoke from cannabis. This may result in detection of analyte(s), e.g., THC, associated with cannabis use. However, the level of this analyte will be significantly lower than the level detected in an actual user. Thus, comparison to the control or the threshold value may be necessary to make an accurate determination as to whether the subject was subject to the “second-hand” smoke of cannabis or was actually using cannabis. In another example, poppy seeds are known to produce a false positive result for heroin. Thus, by comparing the test result with the control can be used to eliminate false positive result of the presence of heroine.

The level of analyte present in the subject can be correlated to a known or “control” value by adjusting (i.e., normalizing) the test result with the total volume of exhaled breath collected from the subject. As expected, higher the volume of exhaled breath collected, the higher the amount of analyte can be detected. By normalizing the test result using the volume of exhaled breath collected, one can eliminate variability due to the volume of exhaled breath. Thus, in some embodiments, the step (iv) of determining the level of drug in the subject's system comprises comparing the normalized amount of the drug metabolite with a control.

Another aspect of the invention provides a method for determining the presence of drug in a subject's system. Such methods include: (i) collecting more than 95% of all aerosol particles from exhaled breath of a subject by having the subject exhale into a breath sample collecting apparatus and measuring the total volume of exhaled breath exhaled into the breath sample collecting apparatus; (ii) determining the amount of a drug metabolite present in the collected aerosol particles; (iii) normalizing the amount of the drug metabolite in the collected aerosol particles based on the volume of exhaled breath; and (iv) determining the presence of drug in the subject's system by using the normalized amount of the drug metabolite determined in said step (iii). In some embodiments, said step (iv) of determining the presence of drug in the subject's system comprises comparing the normalized amount of drug metabolite with a control value.

As discussed above, any breath sample collection apparatus can be used in methods of the invention, as long as the efficiency in collecting aerosol particulates is suitable for accurate and reliable determination. In one particular embodiment, the breath sample collecting apparatus comprises: (a) a flow meter for measuring the volume of exhaled breath collected from the subject; (b) an aerosol collection chamber with a collection surface charged with an electrostatic voltage for collecting aerosol particles from exhaled breath, wherein the aerosol particles are ionized after being exhaled; (c) a conduit for channeling the exhaled breath from the subject to the aerosol collection chamber; (d) an ionizer system in the conduit for ionizing the aerosol particles in the exhaled breath, an extractor system to remove the aerosol particles from the collection surface for analysis; and (e) a pre-collection filter, wherein the pre-collection filter is an ionizing filter connected in fluid-flow relation to the conduit, and the pre-collection filter is positioned in close enough proximity to the aerosol collection chamber to filter ambient aerosols and prevent ambient aerosols from being inhaled by the test subject.

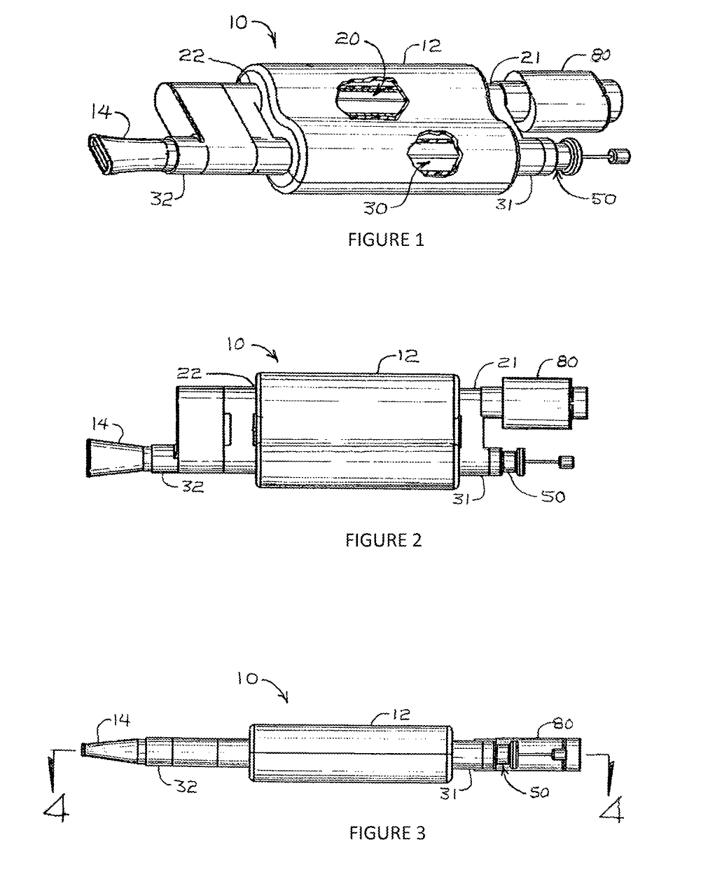

One such a device is disclosed in the present inventors' U.S. Pat. No. 7,364,553. Briefly, the breath aerosol analyte collector 10 is illustrated in FIGS. 1-3. The illustrated device is based on electrostatic particle collection technology and provides a suitable platform for a description of some of the salient features of the invention as well as of certain details that are beneficial, albeit not essential, to the practice of the invention. Other enabling technologies and collector embodiments including, but not limited to, enhanced condensation, are described below. For this electrostatic embodiment 10 as well as other embodiments some or all of the following concepts are used to solve the problems of efficient, effective, reliable, and repeatable exhaled breath aerosol analyte collection: (1) Minimizing or eliminating contamination or skewed results from aerosols in ambient inhaled air; (2) Flow control of exhaled breath to minimize variations in aerosol analyte collection efficiencies, effectiveness, reliability, or repeatability that can result from different flow rates, pressures, time of flow, and the like; (3) Capturing substantially all aerosol materials, including smaller than 100 nm in mean equivalent diameter and preferably as low as 10 nm in mean equivalent diameter, which would include viruses; and (4) Collecting exhaled breath aerosol analytes in concentrations as high as practical for ease of detection, analysis, and other uses.

The example electrostatic breath aerosol analyte collector 10 illustrated in FIGS. 1-3 has a main housing 12 that encloses a pre-collection filter conduit or chamber 20 for removing ambient aerosol from inhaled air and a collection conduit or chamber 30 for removing exhaled aerosol analytes from exhaled breath, as will be explained in more detail below. The collection chamber is a section of the conduit 30 that surrounds the collection rod 40, so collection conduit and collection chamber are sometimes used interchangeably in relation to that section. The first end of the collection chamber is the end where exhaled breath enters the collection chamber and the second end is the opposite end. Upstream means opposite the flow direction of exhaled breath and downstream means the same direction as the flow of exhaled breath. Ambient aerosol as used herein means non-gaseous, air-borne materials in the environment around the test subject and collector, and test subject means a person or animal from which analytes are being collected. A mouthpiece 14 at one end 22 of the collection conduit 30 facilitates a test subject's inhalation of air through the pre-collection filter conduit 20 and exhalation of breath air through the collection conduit 30, although the mouthpiece 14 could be positioned at the end 22 of the pre-collection filter conduit 20 or at a variety of other locations and orientations, as will become apparent to persons skilled in the art, once they understand the principles of this invention. Suffice it to say that inhaled air is drawn through the pre-collection filter conduit or chamber 20, and exhaled breath is directed through the collection conduit or chamber 30, and the mouthpiece 14 or any number of mouthpieces can be positioned at any location or locations that facilitate those functions.

An exhaled aerosol analyte extraction assembly 50 is located at the other end 31 of the collection conduit 30 for extracting aerosol analytes that are removed from the exhaled breath in the collection conduit 30, as will be explained in more detail below. A flow meter 80 is also shown on the breath aerosol analyte collector 10, which can be used to control flow rate of the inhaled or exhaled breath air as well as to provide flow rate measurements used for volume control, collection of aerosol from selected fractions of exhaled breath, and other control functions, as will also be explained in more detail below.

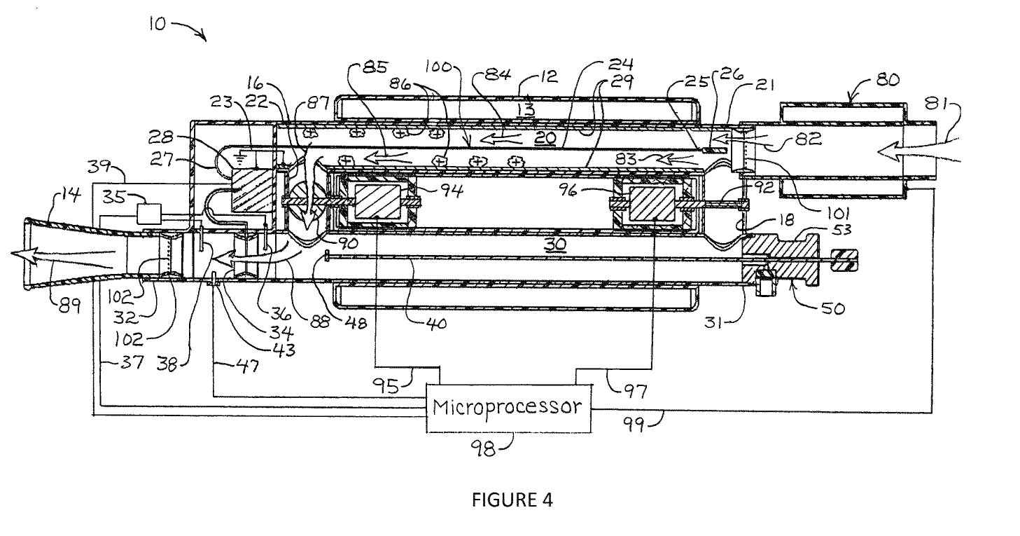

Referring now primarily to FIG. 4 with secondary reference to FIGS. 1-3, air during inhalation of a breath is drawn into the breath aerosol analyte collector 10 through the flow meter 80 and into the inlet end 21 of the pre-collection filter conduit 20, as indicated by the flow arrows 81, 82. The flow meter 80 can be used to measure flow rate or some other flow measuring or flow controlling device can be used in controlling and/or characterizing or quantifying breath flow through the collector 10 for comparing results of collections of exhaled aerosol analytes from one test subject with results from other collections from the same test subject, with results from collections from other test subjects, and with standardized results or quantified indicators of presence or absence of physiological diseases, symptoms, or other problems or concerns. Some flow control can be provided by the test subject in trying to, for example, inhale and exhale in as ordinary a manner as possible during an aerosol analyte collection procedure, but control of the exhale air flow with the collection apparatus 10 itself may provide more consistency, even if the test subject is uncooperative, unconscious, or unable to comply with collection operation instructions. The flow meter 80 as described herein facilitates implementation of such control.

If a flow meter 80 is positioned in another location, which is an option, the air can be drawn directly into the pre-collection filter conduit 20. A pre-collection filter 100, which, in this embodiment 10, is an electrostatic filter but can be any other kind of filter that meets the pre-collection filter performance goals and/or functions described herein, is positioned in the pre-collection filter conduit 20 primarily for the purpose of removing any aerosols in ambient air flowing into the collector 10. The goal is that only exhaled breath aerosols, not aerosols from the ambient air (i.e., ambient aerosols), get collected in the collection conduit or chamber 30, which will be described in more detail below. In other words, if the air inhaled by the test subject contains ambient aerosols, at least some of those ambient aerosols are likely to also be in the exhaled breath and would probably be caught and collected in the collection conduit 30, which is preferably designed and made to collect as much of the aerosol in the exhaled breath as possible in order to collect the analytes from the exhaled breath in sufficient concentrations and quantities to be useable and meaningful.

While many variations and structures of electrostatic and other kinds of filter apparatus are available and can be adapted for use in this invention, the example electrostatic filter 100 in this embodiment 10 comprises a small diameter electric wire 24 (sometimes called a “corona wire”), which extends longitudinally through the pre-collection filter conduit or chamber 20 and is surrounded by an electrically conductive side wall 29 of or on the conduit or chamber 20. The pre-collection filter conduit 20 and the components of the electrostatic filter 100 are preferably sized to introduce little, if any perceivable resistance to the test subject's inhalation efforts, which is one benefit of this single wire 24 design. The wire 24 is anchored at one end 25 to a non-conductive cross-bar 26 and the other end 27 is connected to a high voltage power supply 28. The inside wall of the pre-collection filter conduit 20 comprises an electrically conductive material 29, such as metal (for example, stainless steel), conductive plastic, or other conductive material and is connected electrically to the opposite pole of the high voltage power supply 28. As explained in more detail below, it is preferred, but not essential, that the corona wire 24 be connected to the positive (+) voltage supply terminal, so the conductive wall material 29 is connected to the negative (−) voltage supply terminal, which is often called “ground”, as indicated symbolically at 23. The conductive material 29 can be a separate component, a coating on the wall, or the wall material itself. When the wire 24 is charged with a high voltage, for example, in a range of 2,000 to 12,000 volts, depending on the diameter of the corona wire 24, size of the conduit or chamber 20, and other factors, and the side wall 29 is at opposite in polarity (ground) to the wire 24, it creates corona around the wire 24 that ionizes molecules in the air, which imparts a static electric charge to aerosols in the air that flows, as indicated by flow arrows 84, 85, through the pre-collection filter conduit 20. Consequently, such charged aerosols will cling to the grounded or opposite polarity of the inside wall 29, as indicated in exaggerated scale at 86. The wire 24 is preferably positive, so that ozone production is minimized, although it could be negative, if desired. Consequently, when the air flow, indicated flow arrows 87, 88, 89, reaches the mouthpiece 14 and is inhaled by a test subject (not shown) drawing a breath through the collector 10, it is substantially free of aerosols. Therefore, aerosols collected from the exhaled breath, which will be described below, will include substantially only aerosols introduced by the test subject's lungs and by the airway between the test subject's lungs and lips (not shown). An optional grounded mesh 102 in the end 32 of the conduit 30 just before the air flow is inhaled through the mouthpiece 14 neutralizes any remaining ions and collects any remaining aerosol that did not get captured on the wall 29 of the electrostatic filter 100. It also prevents someone from poking a finger or instrument into the high voltage ionizer assembly 34, which will be described below, and thereby prevent possible damage to the apparatus as well as electric shock to the test subject or other user.

As indicated by the flow arrow 87, a first valve 90, which is illustrated as a butterfly valve in FIG. 4, in the aft cross-over conduit 16 is positioned in a manner that does not impede the flow of air to the mouthpiece 14 during the inhalation of air by the test subject. At the same time, a second valve 92, also illustrated as a butterfly valve, in the fore cross-over tube 18 is closed during inhalation to prevent the ambient air from by-passing the filter 100 in the pre-collection filter conduit 20 by flowing through the collector conduit 30 to the mouthpiece 14.

The first and second valves 90, 92 do not have to be butterfly valves. On the contrary, they could be any of myriad active or passive air control valves, including, but not limited to, one-way, self-actuating check valves, as is understood by persons skilled in the art. However, the butterfly valves 90, 92 have some advantages in that they are simple and inexpensive, yet can be activated for partial closure, full closure, or full open, thus can be used to control flow rate as well as to simply open and close the air flow. In the example of FIG. 4, each butterfly valve 90, 92 is operated by a separate, single-turn brushless actuator or motor 94, 96, such as those manufactured by Saia-Burgess of Murten, Switzerland.

The flow meter 80, as mentioned above, is used to determine the total volume of exhaled breath collected. Such a measurement is important in determining the level of drug present in the subject's system. In addition, the total volume of exhaled breath collected is important for normalization of the result to determine whether the amount or the level of drug in the subject's system meets the threshold value. Flow rate measurements can also provide a number of other benefits. For example, a test subject's inhalation pattern might affect the generation of exhaled breath aerosol. Relevant characteristics of the inhalation pattern may include flow rates, depth of inhalation, time between inhalation and exhalation (e.g., “holding” one's breath), timing and counting number of breaths in a collection period, or exhalation preceding the tested inhalation, pressure variations, or other properties. Flow rates multiplied by time can provide volumes of breaths or fractions of breaths and can be provided by the microprocessor 98 on a real time basis for control of collector functions during inhalation and exhalation as well as being recorded for post-collection analysis purposes. Therefore, data about inhalation flow rates and other patterns, in addition to enabling collector control functions may also enable correction or compensation, or at least explanations for deviations in, analytical data from the collected exhaled breath aerosol analyte specimens.

The flow meter 80 can be a hot wire anemometer or any other flow meter type that measures gas flow rates accurately. If desired, the flow meter 80 can be connected to a microprocessor, illustrated schematically at 98, or any other circuit or device for recording, displaying, or outputting flow rate measurements and/or for controlling the opening, closing, and flow metering functions of the valves 90, 92, as is within the capabilities of persons skilled in the art, once they understand this invention. The actual microprocessor 98, electrical connections 95, 97, 99, and other electric circuits and components can be positioned in the annular space 13 enclosed by the housing 12 or in any other convenient locations.

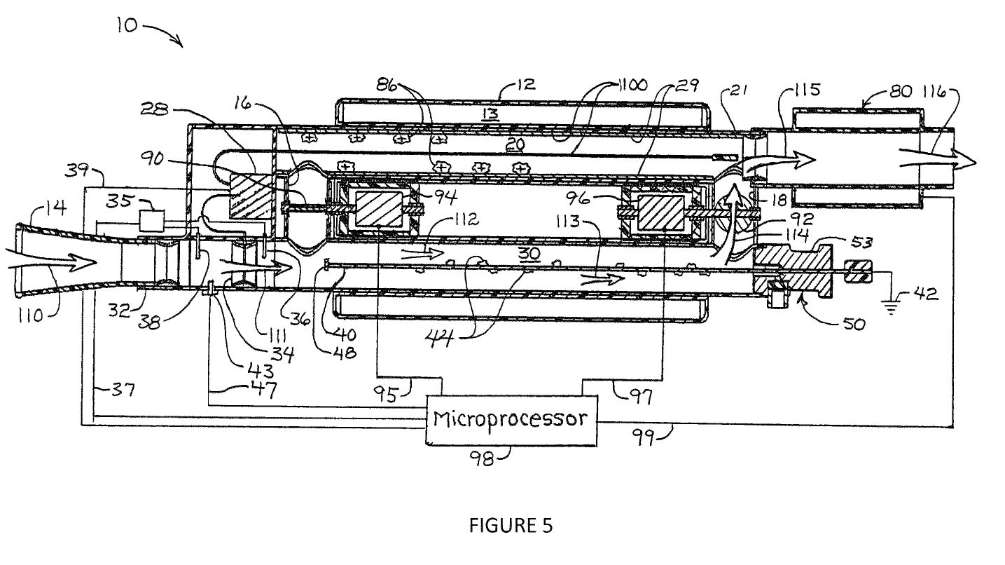

After the breath of air with the ambient aerosols removed is inhaled through the collector 10 by the test subject (person or animal), the test subject exhales the breath into the mouthpiece 14, as indicated by the flow arrow 110 in FIG. 5. In the exhale mode, the first butterfly valve 90 is closed, and the second butterfly valve 92 is opened to allow the exhaled air to flow, as indicated by flow arrows 111, 112, 113, 114, 115, 116, through the collection chamber 30, flow meter 80, and out of collector 10.

Also, in the exhale mode, an ionizer assembly 34 positioned in the collection conduit 30 upstream from a grounded (i.e., negative voltage potential) collection rod 40 is turned on to ionize exhaled air and thereby create electrostatic charges in any aerosols, including analytes in the exhaled breath. The corona wires 104 of the ionizer system 34 (FIG. 9) are preferably connected to the positive (+) terminal of the high voltage power supply 28, so, as explained above, the term “grounded” for the collection rod 40 means it is connected electrically to the negative (−) terminal of the power high voltage power supply 28, as indicated by the “ground” symbol 42. Again, since practically all of the ambient aerosol 86 was removed from the inhaled air in the pre-collection filter 100, as explained above, substantially all of the aerosols in the exhaled air are derived from the test subject's lungs and airway. As the positive charged, ionized air flow 112, 113 from the ionizer system 34 continues through the collection tube 30, the airborne, positive charged aerosols from the test subject's lungs flow past the collection rod 40, which extends from the extraction assembly 50 toward the ionizer assembly 34. As mentioned above, the collection rod 40 is at negative (−) potential (i.e., grounded, as indicated by the ground symbol 42, so the positive charged aerosol are attracted to, and cling to, the negative charged collection rod 40, as illustrated in somewhat exaggerated sizes at 44. Preferably, most, if not all, of the aerosol analytes in the exhaled breath are collected on the collection rod 40 before the exhaled air flows out of the collection chamber 30. The longer the collection conduit 30 and rod 40, and the slower the exhaled air flow through the collection conduit 30, the more complete the aerosol analyte removal from the exhaled air will be. Therefore, it may be desirable to control the velocity or flow rate (volumetric and/or mass flow rate) of exhaled air flow 112, 113 through the collection conduit 30 as well as the volume of exhaled air for accuracy and efficiency as well as for standardization, reliability, reproducibility, and other purposes. In the example collector 10, flow rate measurements by the flow meter 80 can be fed by the connection or link 99 to the microprocessor 98 for use in adjusting the valve 92 in the fore cross-over conduit 18 to maintain the exhaled air flow velocity or flow rate in the collection conduit 30 in a desired range.

There is no significant detriment to lack of moisture in electrostatic collection of aerosol particles, so there is no need for provisions in collector 10 to maintain humidity in the exhaled air before and during collection of aerosol on the collection rod 40. In fact, there are advantages to dryer airflow and dryer aerosols for electrostatic collections, so it may be desirable in some applications to add some kind of dryer, such as a heater (not shown) to the collector 10, for example, between the grounded mesh assembly 102 and the ionizer assembly 34 to dry the exhaled air and aerosols before undergoing the electrostatic aerosol collection.

Any desired number of breaths can be exhaled by the test subject through the collector 10 as the exhaled breath aerosol analytes are collected on the collection rod 40. If the valves 90, 92 are of a type that have to be driven from closed to open positions and vice versa, as opposed to self-actuated, one-way check valves, some kind of sensor may be used to facilitate actuation of the valves 90, 92 to open and close the cross-over conduits 16, 18 as required to direct inhale air flow through the pre-collection filter conduit 20 and to direct exhaled air flow through the collection conduit 30. While myriad sensor systems would work for this purpose, the collector 10 is illustrated, for example, with a pair of ion detectors 36, 38 positioned on opposite sides of the ionizer assembly 34. The second ion detector 38 is grounded. If there are ions in the air flow that contacts the first ion detector, a current can be detected by an ammeter 35 or other suitable detector. The ionizer assembly 34 can be at least at a low level that produces enough ions in the air flow to be detected by the ion detectors 36, 38. Because most, if not virtually all of the ions in the air flow through either conduit 20 or conduit 30 get eliminated by the grounded components 29, 40, air flow past the first ion detector probe 36 during inhalation will produce little or no current at ammeter 35. This condition can be used to indicate inhalation and, for example, can be communicated to the microprocessor 98 via connection or link 37 for use in generating control signals on links 95, 97 to the valve actuators 94, 96 to open valve 90 and close valve 92 for the inhalation mode, i.e., to direct inhalation air through the pre-collection filter conduit 20 and not through the collection conduit 30. Conversely, when air is being exhaled by the test subject, air flow through the ionizer assembly 34, as indicated by flow arrow 111 in FIG. 5, causes ionized air to contact the ion detector problem 36 to produce a current at ammeter 35. This condition can be communicated to the microprocessor 98 or other suitable circuit to activate the exhale mode, i.e., to close the valve 90 and open the valve 92 to direct exhaled air flow through the collection conduit 30 and not through the pre-collection filter conduit 20. The microprocessor 98 can also communicate via a link 39 to an appropriate circuit associated with the high voltage power supply 28 to turn up the power on the ionizer assembly 34 during exhaled breath for better aerosol analyte collection during exhalation and to turn down the power on the ionizer assembly 34 during inhalation.

As mentioned above, because exhaled breath aerosols are few and difficult to collect, analyze, quantify, characterize, and standardize, it is helpful to collect them in the highest practical concentrations. As also mentioned above, the first one-third to one-half of a typical exhaled breath is reflux of inhaled air that never reaches the lungs where alveolar gas exchange occurs and aerosol analytes of interest are produced. Therefore, it is known, for example, that carbon dioxide exchanged during respiration appears at highest concentrations in the later fractions of an exhalation, and it is quite probable, albeit not yet proven, that higher concentrations of exhaled breath aerosols are also highest in the later fractions of exhaled breaths. Consequently, it may be desirable to have the capability of starting collection of exhaled breath aerosol only when the later fractions of the exhaled breaths pass through the collection conduit or chamber 30.

This kind of collection procedure can be implemented in a number of different ways. For example, it can be done by manually turning on the electrostatic collection components, e.g., the ionizer system 34, for the collection conduit or chamber 30 only after a first fraction (e.g., one-third to one-half) of the exhaled breath has been released. It can also be accomplished by turning on the same components with a timer, for example associated with the microprocessor 98, after a preset time has elapsed from detection of the start of an exhalation. A similar effect can be attained by delaying the opening of the second valve 92 and closing the first valve 90 to prevent collection of aerosol from the first fraction of the exhaled breath on the collection rod 40. Volume, calculated with flow-rate measurements and time, can also be used as an input criteria, either alone or with other input data or criteria to control the collector 10 component functions for this purpose. Another approach (not shown) may be to provide another outlet port from the collection conduit or chamber 30, such as a lateral side port, along with a valve that can be opened when the marker, e.g., carbon dioxide, level is below the desired collection concentration level or threshold to simply vent the first portion or fraction of the exhaled breath out of the system until the marker level rises to a threshold at which collection of aerosol is desired. However, a more precise and automated system for collection of exhalation from a more aerosol-rich fraction of the exhaled breath, instrumental sensing of a suitable marker in the exhaled breath, for example, but not for limitation, carbon dioxide, can be used to start and/or stop certain collection components, such as the ionizer system 34 or valve actuators 94, 96, a valve (not shown) to vent the first fraction of the exhaled breath out of the system until the marker rises to a desired level or concentration for collection, or the like. Therefore, for example, a carbon dioxide detector 43 is shown near the entrance end 32 of the collection conduit 30 for sensing concentration of the carbon dioxide in exhaled breaths for use in starting exhaled breath aerosol collection only after carbon dioxide concentrations reach some predetermined threshold level. The carbon dioxide detector 43 can be connected to the microprocessor 98, as indicated schematically by link 47, if desired so that the threshold and responsive functions can be processed and controlled, as is within the capabilities of person skilled in the art, once they understand the principles of this invention. A suitable carbon dioxide detector for this purpose may be, for example, a respiratory capnometer, such as the model V8200 manufactured by Harvard Apparatus of Hollister, Mass., or any other carbon dioxide detector operated on a suitable circuit as is within the capabilities of persons skilled in the art.

As can be seen from the example exhaled breath aerosol collector 10 described above, it implements one of the principles of improved exhaled breath aerosol collection according to this invention, i.e., identifying a property of exhaled breath aerosol that can be enhanced to become more responsive to application of a force that enables improved collection and then applying such a force to the exhaled breath aerosol. In the electrostatic collection example of collector 10, the property, a possible electrostatic charge of some of the aerosols, is enhanced to a strong and more uniform electrostatic charge of known polarity for most, if not all, of the exhaled breath aerosol particles and/or droplets, which can be accomplished by surrounding the aerosol particles and/or droplets with charged ions which impart charges to the exhaled breath aerosol and then applying electrostatic force to collect the aerosol particles and/or droplets on the collection rod 40.

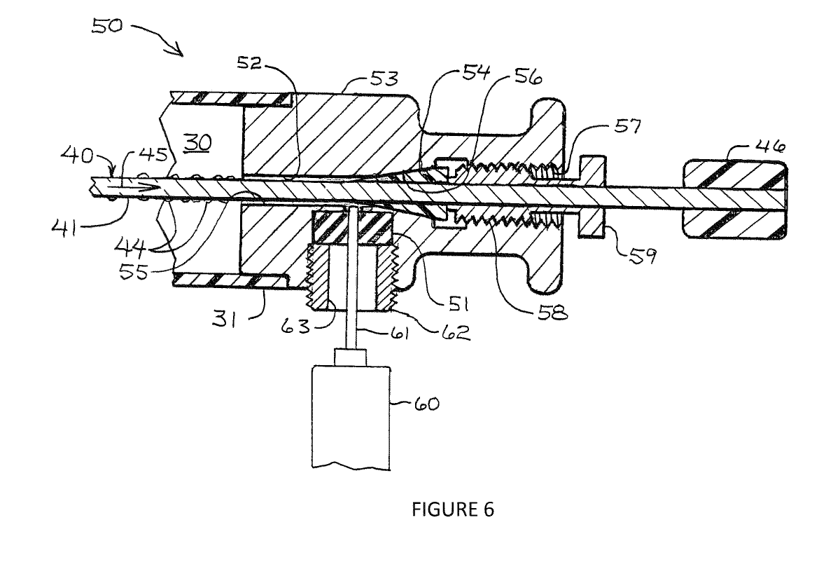

When the predetermined number of breaths or other desired criteria, such as volume of breath processed by the collection chamber at a desired or regulated flow rate, have been met to terminate the exhaled breath aerosol analyte collection, the collector 10 can be removed from the mouth of the test subject to extract the collected aerosol analytes for further processing and/or analysis. Again, there are myriad ways that such extraction can be done, but the collector 10 described above has an extractor assembly 50 at one end of the collection conduit, as shown in FIGS. 1-5. The extractor assembly 50 is best seen in FIG. 6, which is an enlarged cross-section of the extractor assembly 50 similar to the cross-section in FIGS. 4 and 5.

Essentially, to extract the exhaled aerosol analytes 44, which are captured on the surface 41 of the collection rod 40, as described above, a blunt needle 61 of a syringe 60 is pushed through a septum 51 to inject just enough liquid solvent to fill an annular space 52 around the rod 40 in the body 53 of the extraction assembly 50. The liquid solvent will usually be a kind of high purity water, such as high performance liquid chromatography (HPLC) grade water, although other suitable solvents can be used, for example, but not for limitation, any of a number of buffer solutions that are widely used in bio-chemical analysis techniques and procedures. If desired, the collector 10 can be turned and held with the bore 55 in the body in a vertical orientation during this extraction phase so that gravity helps to retain the liquid solvent in the annular space 52, although capillary action may be sufficient to retain the liquid solvent in the space 52 in other orientations. Then, the collection rod 40 is pulled longitudinally through a seal 54, as indicated by arrow 45, which wipes or scrapes the analytes 44 off the surface 41 of rod 40, where they are retained and dissolved into the liquid solvent in the annular space 52.

When enough of the rod 40 has been pulled through the seal 54 to wipe or scrape substantially all of the analytes 44 off the rod surface 41, the solvent along with the dissolved analytes can be drawn by the syringe 60 out of the space 52. The analytes can then be recovered from the solution in the syringe 60 by conventional laboratory or commercial processes for whatever further analysis or study is desired. An optional limit stop, such as a flange 48 (FIG. 5), can be provided on the end of collection rod 40, if desired, to prevent accidental removal of the rod 40 from the body 53 of the extractor assembly 50.

As illustrated in FIG. 6, the extraction assembly can be made with an initial axial bore 55 extending longitudinally through the body 53 with a diameter that is large enough to leave the annular space 52 between the collection rod 40 and the body 53, when the rod 40 is positioned in the bore 55. The bore 55 then widens in the mid-section of the body at 56 to accommodate the seal 54. The seal 54 and corresponding widened bore 56 can be cylindrical or any other convenient shape, but a preferred shape is tapered or conical, as illustrated in FIG. 6, to accommodate uniform snugging of the seal 54 onto the rod 40 for an effective seal against solvent leakage and for effective wiping or scraping of the analytes off the surface of the rod 40. A distal end portion 57 of the bore can be threaded to receive a threaded gland 58 for tightening and retaining the seal 54 in place. The more the gland 58 is tightened against the seal 54, the more the tapered surface of the bore section 56 squeezes the seal 54 against the rod 40. The seal 54 can be made of any of a number of suitable materials, such as PEEK® (polyaryletherketone), which is available from Upchurch Scientific, of Oak Harbor, Wash. PEEK® is preferred because of its strength, rigidity, chemical and physical inertness, high dielectric strength as an insulator, and compatibility with sterilization techniques. A flange 59 on the distal end of the gland 58 can be shaped to accommodate a tool, such as a wrench (not shown) for tightening, and it can serve in combination with a knob 46 on the end of the collection rod 40 as a limit stop to limit longitudinal movement of the rod 40 into the conduit 30. The collection rod 40 can be made of stainless steel or other suitable electrically conductive material, and it is preferred to have a surface roughness of no more than 200 nanometers so that the seal 54 can effectively wipe or scrape the small analyte particles 44 off the rod surfaces. Longitudinal, rather than radial scratches or roughness is also helpful in this regard, although any scratching or roughness is preferably minimized as much as practical.

The septum 51, which is preferably resilient elastomeric or flexible latex or some other resilient material that accommodates puncturing by the needle 61 and that will seal around the needle 61 to prevent leakage and reseal itself when the needle is removed, can be held in place in a transverse bore by a hollow gland 62 screwed, as shown in FIG. 6, or glued or friction held (not shown) in the body 53. The hollow bore 63 in the gland 62 accommodates insertion of the needle 61 into the septum 51. If desired, the septum 51 can be pre-split to accommodate insertion of a blunt needle 61. Also, a valve, such as those used in intervenous connections could be used in place of the septum 51.

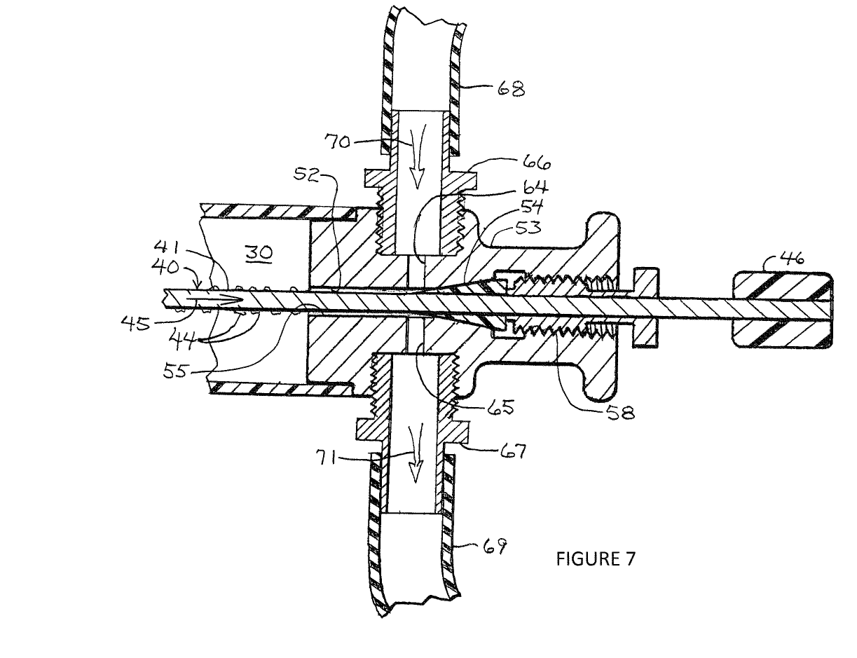

An alternative to the septum 51 and syringe 60 can be the arrangement shown in FIG. 7, wherein there are two conduits 64, 65 extending radially in different directions from the bore 55. A pair of fittings 66, 67 fastened to the body 53 in alignment with the conduits 64, 65 connect tubes 68, 69 to the respective conduits 64, 65, so that the liquid solvent can be flowed transversely, as indicated by arrows 70, 71 through the bore 55 adjacent the seal 54 as the collection rod 40 is drawn through the seal 54 or after the rod 40 is drawn through the seal 54. As the analytes 44 are scraped or wiped off the surface 41 of the collection rod 40, the solvent flow 70, 71 dissolves them and carries them through the downstream tube 69 to any suitable receptacle or process (not shown), where they can be recovered by conventional techniques for further analysis, classification, or study.

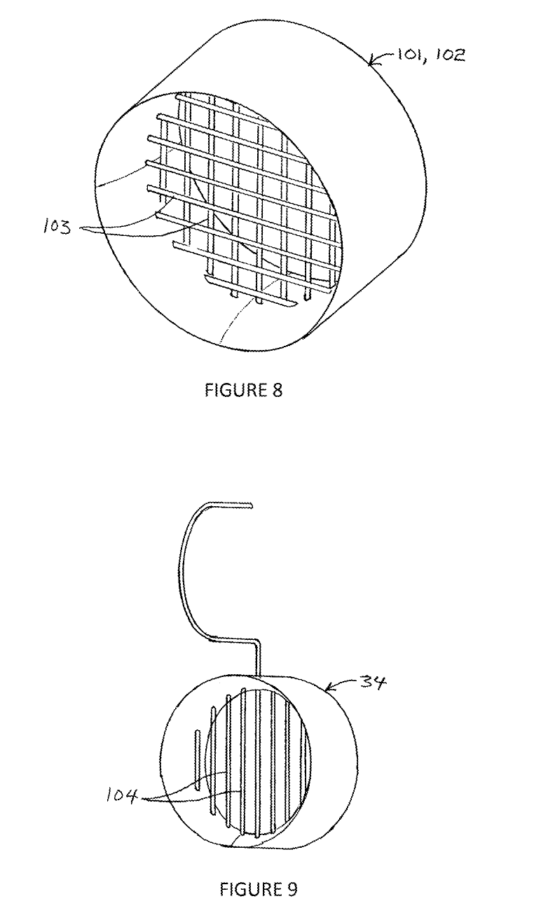

Referring again primarily to FIGS. 4 and 5, a first grounded mesh assembly 101 is positioned at the entrance to the pre-collection filter conduit 20 and a second mesh assembly 102 is positioned at the entrance to the collection conduit 30. These grounded mesh assemblies 101, 102 prevent a person from inserting an object or finger into the high voltage ionizer elements 24, 34, respectively. They can also stop large particles, such as dust, insects, food particles, saliva, sputum, expectorate, and the like from entering the conduits 20, 30. Generally, these and other artifacts, which are larger than about 10 microns mean equivalent diameter are prevented from entering the collection chamber by the mesh assembly 102 or by any other convenient trap or device. An example grounded mesh assembly 101, 102 as shown in FIG. 8 (not to scale), and an example ionizer assembly 34 is shown in FIG. 9 (not to scale). Both are made of electrically conductive materials. The screen 103 of the mesh assembly 101, 102 can be, for example, 100 mesh fabricated with 500 micrometer tungsten or stainless steel wire, which conveniently has no more than 10% blockage of flow area through the screen 100, which may be desirable so that the test subject does not feel significant resistance by the collector 50 to exhalation effort, but is not a requirement. Of course, the flow regulation provided by the flow meter 80, microprocessor 98, valves 90, 92, and other components may present some resistance to exhalation by the test subject, especially if the test subject tries to exhale too rapidly or otherwise outside the breath flow criteria applied by these components for accuracy, reproducibility, standardization, comparability, and the like. The ionizer 34 can comprise a plurality of small diameter tungsten wires 104 (e.g., 250 micrometers) positioned parallel to each other and perpendicular to the air flow 88 (FIG. 5). They are raised to a positive potential sufficient to produce an ionized field in air, for example, 2,000 to 12,000 volts, or about 70 kV/m. The positive potential for the ionized air flow is preferred over negative to reduce ozone production, but negative may be more useful for some applications.

There are many other possible variations that can be devised to practice this invention. For example, but not for limitation, the inhaled air and exhaled air do not have to be routed through the same flow meter 80, which is optional, or even through the same entrance end 21. In fact, the pre-collection filter conduit 20 and the collection conduit 30 could be separate, each with its own respective mouthpiece, which would simply require the test subject to inhale from one of the mouthpieces through the separate pre-collection filter conduit or chamber 20 and then to exhale through the other of the mouthpieces into the collection conduit. While this maneuver would add a slight complexity for the test subject, it could eliminate the valves from the apparatus and still accommodate practicing the invention. Also, such mouthpieces 14 can have any convenient shape or structure other than that shown in FIGS. 1-5, such as a face mask with a breath port, an endotrachial tube, or any other device for capturing the air flow of a test subject's breath and channeling it through components in collector 10.

Also, as mentioned above, there are many possible valve variations that can be used to practice the invention. For example, but not for limitation, the electrostatic collection rod 40 could be replaced with any other shape or apparatus that will collect the charged aerosol analytes and from which such analytes can be recovered to practice this invention. Also, the butterfly valves 90, 92 could be mounted on a common shaft, but rotated 90 degrees in relation to each other, and actuated by one actuator or motor. In such an arrangement, rotation of the shaft in one direction would open one valve 90 as the other valve 92 is closed, and vice versa. Also, the valves could be operated manually. Such manual operation would add some complexity for the user, but the apparatus would be less complex and less expensive. On the other hand, further automation can be added to practice the invention. For example, but not for limitation, the collection rod 40 could be a continuous wire drawn automatically through the collection chamber 30 and extraction assembly 50, especially in combination with the continuous solvent flow 70, 71 of the alternate embodiment shown in FIG. 7.

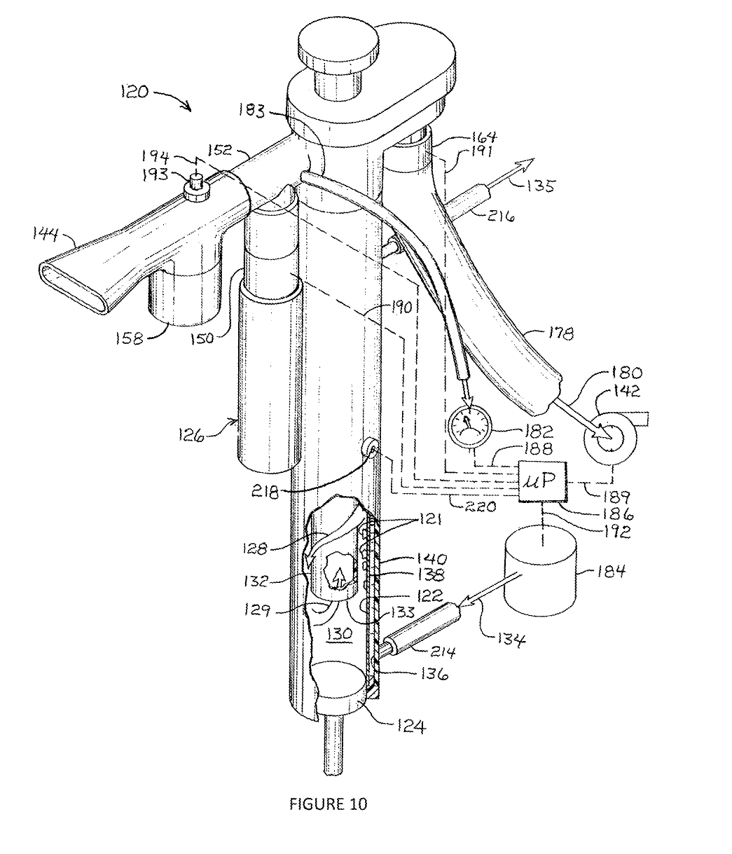

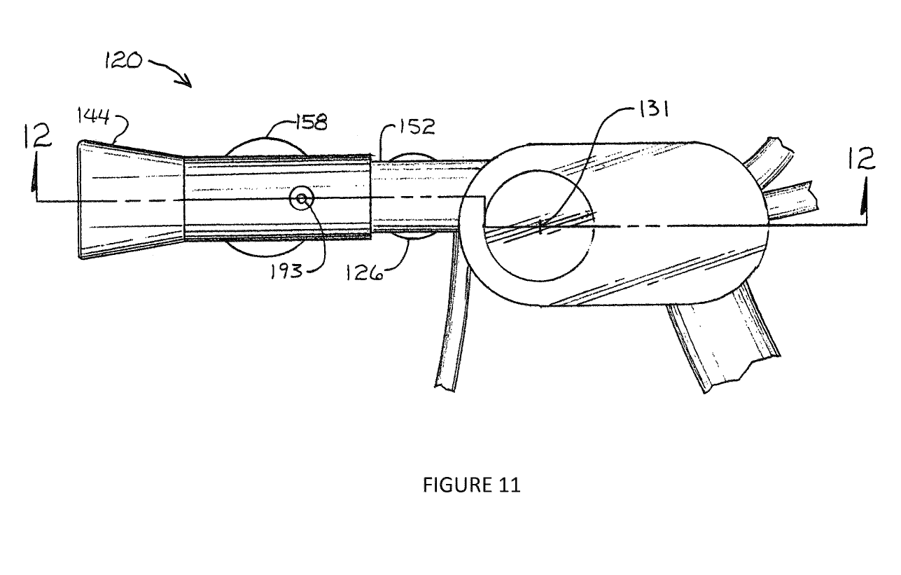

Another example breath aerosol analyte collector 120, illustrated in FIGS. 10-12, enhances a different property of the exhaled breath aerosol, its mass, and then applying centrifugal force to the aerosol to facilitate collection of the exhaled breath aerosol analytes. More specifically, in this embodiment breath aerosol analyte collector 120, the conditions are created to enhance condensation of the water vapor in the exhaled breath on the aerosol particles and/or droplets to increase the mass of the aerosol, as will be explained in more detail below, and then applying centrifugal force to the aerosol with enhanced mass to enhance collection of the aerosol 121 on a condensation surface 122, as will also be described in more detail below. Then some extraction means, for example, the wiper 124, is used to extract the collected aerosol 121 from the collection surface 122, as will also be explained in more detail below.

Essentially, ambient air is preferably inhaled by the test subject (not shown) through a pre-collection filter assembly 126 to remove any ambient aerosols for the reasons explained above. Then, the breath or air is exhaled by the test subject through flow constriction, such as a jet nozzle or orifice (explained below), to create a jet stream flow into an expansion chamber and/or collection chamber (explained below) to expand, cool, and cause condensation of water vapor in the exhaled breath, and to create a spiral flow of the exhaled breath, indicated by flow arrows 128, through the collection chamber 130 to force aerosol against the tubular collection surface 122. The aerosol in the expansion chamber nucleates the water vapor condensation to add mass, as will be explained in more detail below. The spiral flow 128 creates centrifugal forces on the aerosol and condensed water, and it creates turbulences that help to break down boundary layers of fluid flow on the collection surface 122. Both of these effects enhance probability that the aerosols and condensed water in the exhaled breath will contact and be retained by the collection surface 122, as illustrated in exaggerated scale at 121. The exhaled air flow, stripped of most, if not all, of the exhaled aerosols then turns as indicated by flow arrow 129 and exhausts out of the collection chamber 130 through an exhaust tube 132, which extends longitudinally through the collection chamber 130, where the tube 132 also helps to shape and maintain the spiral flow 128. An optional cooling fluid 134 can be flowed through a space 136 between the collection tube 138 and outer shell 140 to help maintain the collection surface 122 in a desired temperature range for efficient condensation and collection of water and aerosols 121 on the collection surface 122. It is preferred, but not essential, that the exhalation of the breath be assisted by a vacuum pump 142 in order to help maintain enough of a pressure drop to enhance nucleation and condensation through the jet nozzle or orifice (explained below) without extraordinary exhaling effort by the test subject. The exhaust tube 132 is removable from the collection chamber 130 to accommodate extraction of the collected aerosols 122 by pushing the wiper 124 longitudinally through the collection chamber 130.

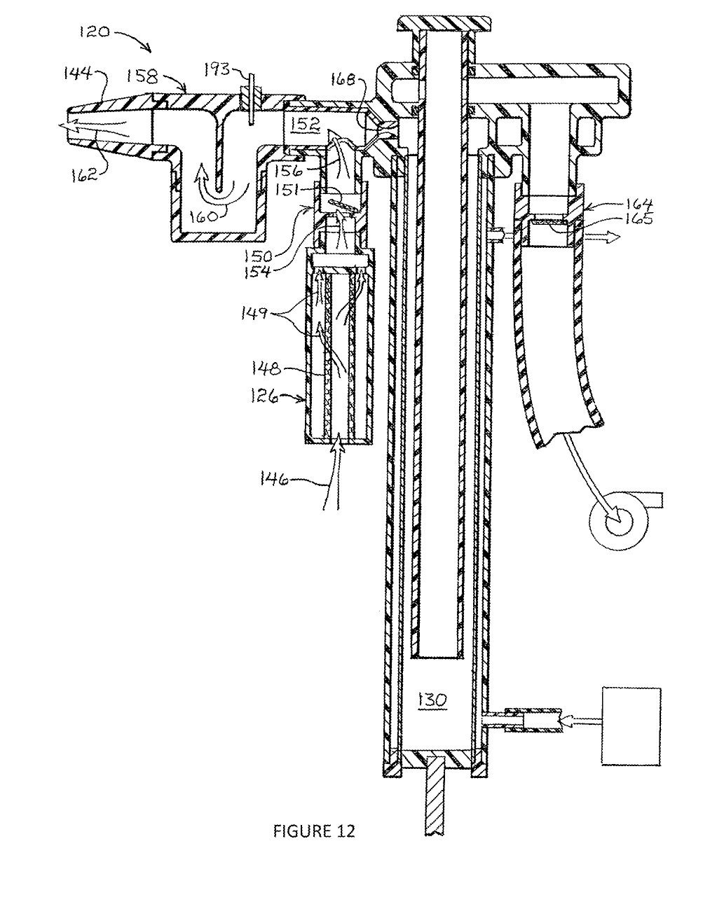

With reference now primarily to FIG. 12 along with secondary reference to FIG. 10, a mouthpiece 144 is provided for the test subject to inhale and exhale breaths through the aerosol analyte collector 120. Again, the mouthpiece 144 can have any shape or structure and can be part of a face mask (not shown), an endotrachial tube or any other device for capturing a test subject's breath and channeling it through components in the collector 120. Inhalation of a breath draws ambient air through the pre-collection filter assembly 126, as indicated by flow arrow 146 to remove ambient aerosols from the air being inhaled so that any aerosol analytes 121 collected on the collection surface 122 will be derived from the test subject's lungs and airway and will not be contaminated by ambient aerosols. The pre-collection filter assembly 126 is depicted for example in FIG. 12 as having a paper or cloth filter element 148 to catch ambient aerosols, but any other kind of filter technology or apparatus that is effective to catch and remove ambient aerosols from the air being inhaled can also be used for this purpose. A suitable pre-collection filter 126 for this purpose may be, for example, an Air Life™ Bacterial Viral Filter, manufacturer's part no. 001851, available from Cardinal Health, Inc. of Dublin, Ohio.

The air flows as indicated by arrows 149 through the filter assembly 126, through a first one-way check valve assembly 50 or any other suitable valve type, and into the main air duct 152, as indicated by flow arrows 154, 156. An example one-way check valve that will work for this purpose is part no. 1664 “one-way valve” available from The Hudson RCI Company, of Temecula, Calif. From the main air duct 152, the air flows backward through a classifier or trap 158, which will be explained in more detail below, and through the mouthpiece 144, as indicated by arrows 160, 162, to be inhaled by the test subject (not shown). The mouthpiece, trap, main air duct, and connecting sections are sometimes jointly or severally referred to herein as a conduit. The first valve assembly 150 opens during inhalation, as depicted diagrammatically by the open valve member 151 to allow inflow of air through the filter assembly 126, while a second one-way check valve assembly 164 closes, as indicated by the closed valve closure member 165, to prevent backflow of air through the collection chamber 130 during inhalation. Because of the small size of the jet nozzle or orifice 168, which will be explained in more detail below, the second one-way check valve may not be needed. However, if a valve 164 is needed to prevent backflow, it can be any suitable valve type to perform that function, not just a one-way check valve.

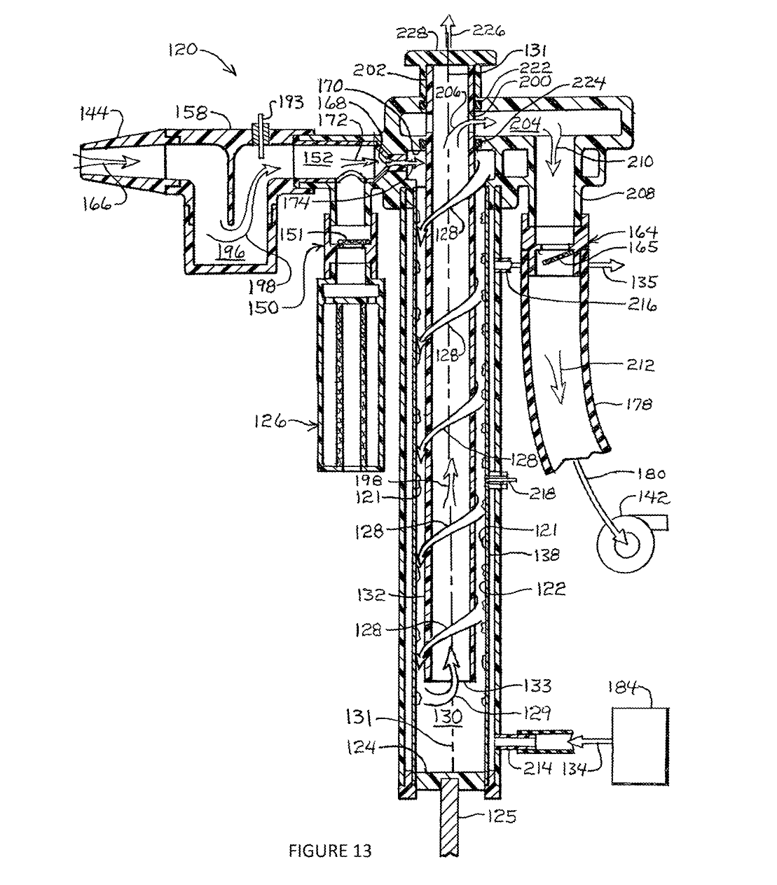

Next, after inhalation as described above, the test subject exhales breath, which flows through the collector 120, as best seen in FIG. 13 with continuing secondary reference to FIG. 10. As shown in FIG. 13, the exhaled breath enters the collector 120 through the mouthpiece 144, as indicated by flow arrow 166. Upon this reversal of airflow from inhalation to exhalation, the first valve assembly 150 closes, as indicated diagrammatically by the closed valve member 151, and the second valve assembly 164 opens, as indicated diagrammatically by the opened valve member 165. This reversal of valve assemblies 150, 164 prevents the exhaled air from flowing backward through the pre-collection filter assembly 126 and directs the flow instead from the main air duct 150 through the nozzle, orifice or other flow constrictor 168 and into the expansion chamber 170 and/or collection chamber 130, as indicated by flow arrows 172, 174. As mentioned above, an optional vacuum source can be connected to an exhaust outlet conduit 178, as indicated diagrammatically by vacuum pump 142 and arrow 180, to increase and/or maintain an adequate pressure drop across nozzle 168, i.e., pressure differential between the main air duct plenum 152 and the expansion chamber 170 to get the desired cooling and nucleated condensation effect in the expansion chamber 170 without requiring extraordinary exhaling effort by the test subject. An optional pressure transducer 182 or pressure conduit 183 to such a pressure transducer (illustrated diagrammatically by pressure transducer 182 in FIG. 10) can be tapped into the main air duct plenum 152 to sense the build-up of pressure in the plenum 152 upon the start of exhalation by the test subject for any of a number of control functions. Another pressure sensor (not shown) can be tapped into the expansion chamber 170 and/or collection chamber 130 to monitor pressure in these chambers 130, 170 or pressure drop across the jet nozzle 168 for feedback control to the vacuum source 142 to increase or decrease the pressure in the expansion chamber 170 and/or collection chamber 130 as needed for the desired amount of jet cooling effect. Temperature sensors (not shown) can also be added in the plenum 152 and expansion chamber 170 for achieving the desired gas temperature differentials for a good balance between enough nucleated condensation for good exhaled breath aerosol collection without too much condensate that dilutes the collected analyte specimens. For example, the pressure increase in plenum 152 from the start of exhalation can be used to activate the vacuum source 142, to activate the cooling fluid source 184, to actuate valve 150, 164 (if they are of a type that require motive force for activation), and myriad other functions that may occur to persons skilled in the art, once they understand the principles of this invention. A microprocessor 186 can be used to facilitate these and other functions, as illustrated schematically by phantom lines 188, 189, 190, 191, 192 in FIG. 10, or by analog or other methods. Such implementations are well within the capabilities of persons skilled in the art and need not be described here for an understanding of this invention. Likewise, a number of other sensor and/or transducer technologies, such as flow meters, manual switches, and others can be used to implement these functions, as will also be understood by persons skilled in the art, once they understand the principles of this invention. For example, a carbon dioxide sensor 193 can be used to detect increase in carbon dioxide, which may indicate exhaled breath to start one or more of the functions of the collector 120. The link 194 to the microprocessor 186 in FIG. 10 is a schematic indication of control functions based on carbon dioxide detection in the air flow. One particular advantage of a carbon dioxide detector 193 is that it can distinguish between exhaled air that has been no deeper than the test subject's airway, which has near normal air content of carbon dioxide, from air that is exhaled from deep in the lungs, which has higher carbon dioxide content. Thus, for example, if the valves 150, 164 are actively controllable or actuatable, as opposed to self-actuating one-way check valves, they can be switched on or off to allow exhaled air to flow into the collection chamber 130 only when an increase in carbon dioxide indicates that breath exhaled from deep in the lungs has reached the collector 120. If it is determined that part or all of an exhaled breath is not to be accepted in the collection chamber 130 for this reason or for any other control reason (e.g., insufficient velocity or flow rate, volume control, etc.), the exhaled flow can be directed back through the pre-collection filter assembly 126 to the atmosphere, or another outlet port and valve (not shown) can be provided anywhere upstream of the jet nozzle 168 for redirecting the exhaled air out of the collector 120. For example another outlet port and valve (not shown) could be connected into or out of the main plenum 152 or the valve 150 could be a 3-way valve connected to another outlet port to divert such unwanted flow out of the system, if it is preferred to avoid such backward flow through the filter assembly 126. Of course, the microprocessor 136 or other control systems used can reverse those functions discussed above when the pressures, flows, carbon dioxide, temperatures, and the like reverse or get out of desired ranges for the functions.

Referring again primarily to FIG. 13 with secondary reference to FIG. 10, the exhaled breath 166 is preferably directed first through a classifier or trap 158 to stop and retain any large materials or artifacts (e.g., greater than about 10 microns mean equivalent diameter) in the exhaled air, such as bits of food, sputum, expectorate, saliva, and the like, which could skew collection and/or measurements of collected aerosol analytes of interest. The trap 158 in FIG. 13 is illustrated, for example, as a simple U-shaped air conduit 196, in which such large materials would be trapped, because they would have too much mass to make the U-turn illustrated by flow arrow 198 and defy gravity to get into the main air duct plenum 150. However, many other types of traps or classifiers would also work for this purpose.

As mentioned above, from the main air duct plenum 152, the air flow 172, 174 is directed through a jet nozzle or orifice 168 into the expansion chamber 170 and/or collection chamber 130. The jet nozzle or orifice 168 (not drawn to scale) is very much smaller in diameter than the plenum 152, so air flow through the jet orifice accelerates to a high velocity and then escapes in a jet stream into the lower pressure expansion chamber 170. The result of this effect is an adiabatic expansion and cooling of the fluid as it expands into the lower pressure expansion chamber 170, which causes super-saturation of water vapor in the stream of exhaled breath.

Water vapor in a rapidly cooling, super-saturated volume of carrier gas condenses upon solid and/or liquid aerosols suspended in the air flow, i.e., on the exhaled breath aerosols, which nucleate the condensation. Of course, condensation also occurs on the interior walls of the expansion chamber 170 and on the interior surface 122 of the collection chamber 130. However, a significant feature of this implementation of the invention is the creation of conditions that enhance such nucleated condensation on the exhaled breath aerosols, which adds mass to the aerosols and, thereby, renders them more susceptible to a collection force.

One of the collection forces used in this implementation of the invention is centrifugal force applied to the aerosols, which has a greater effect on the aerosols that, along with condensed water on them, have increased mass. The centrifugal force is applied in this embodiment 120 by directing the jet stream flow 174 tangentially, or offset from the longitudinal axis 131 of the collection chamber 130, into the expansion chamber 170, which, along with the low pressure created by the vacuum source 142, causes a vortical stream of the exhaled breath spiraling down the annulus between the exhaust tube 132 and the collection tube 138, as indicated by flow arrow 128. The collection chamber 130 is preferably in the shape of a figure of revolution, such as a cylinder, with a longitudinal axis 131, and the jet stream flow 174 is directed in offset relation, to the longitudinal axis 131 into the expansion chamber 170, which is merely a top part and/or top extension of the collection chamber 130. The components of the main air duct or conduit 152 intersecting the expansion chamber 170 and/or the collection chamber in a tangential manner with or without the constriction or nozzle 168 are sometimes referred to as a vortex generator. The vacuum source 142 is not essential, because the exhaled breath in the plenum 152 itself raises the pressure in the plenum above the pressure in the expansion chamber 170 and collection chamber 130, but the vacuum source 142 enhances this process and reduces the feeling of resistance to exhalation felt by the test subject. The resulting vortex 128 creates a powerful centrifugal force on the aerosol suspended in the vortical stream 128, especially those aerosols that are laden with the additional mass of the nucleated condensation, as explained above. The dwell time of the exhaled breath stream 128 in the collection chamber 130, i.e., the amount of time that it takes for an average air molecule to spiral down the vortex 128 from the expansion chamber 170 to the entrance 133 of the exhaust tube 132, depends on dimensions of the collection chamber 130 and operating pressures in the collector 120, but the centrifugal force acts on any aerosols in the vortical stream 128 all the way down the annular space to the exhaust tube entrance 133. These centrifugal forces accelerate the particles toward the condensation surface 122 of the collection tube 138. The more mass an aerosol has, the more it is accelerated toward the collection surface 122.

As mentioned above, there is also some condensation of water vapor from the exhaled breath on the collection surface 122, depending on the temperature difference between the water vapor in the exhaled breath and the collection surface 122. However, this implementation of the invention requires only enough difference in temperature between the water vapor entering the expansion chamber 170 and the temperature in the expansion chamber 170 and continuing into the collection chamber 130 (the expansion chamber 170 is merely an upper portion and/or extension of the collection chamber 130) to enable mass accretion on aerosol particles and droplets by nucleated condensation to assure capture of a consistent and majority of aerosol particles and droplets on the collection surface 122. Further increase in that temperature differential will only increase condensation of water vapor directly on the collection surface 122 and, thereby, increase dilution of the captured exhaled breath aerosol analytes on the collection surface 122 by the additional condensed water on the collection surface 122 without proportionally increasing the collected quantity of aerosol analytes. Therefore, to enable consistency and repeatability of aerosol analyte collection that can be analyzed and/or compared in a meaningful manner to other aerosol analyte collections from the same test subject and/or from other test subjects or to standards and the like, it may be important to maintain the temperature of the collection surface 122 and collection chamber 130 within a desired or prescribed temperature range. Therefore, a temperature control system may be desirable and, in the example collector 120, is illustrated as a temperature controlled cooling fluid jacket or chamber 136 between the collection tube 138 and an outer shell 140 to maintain a temperature controlled collection surface 122. The cooling fluid can be circulated from a source 184 through an inlet tube 214 into the jacket 136 and out from the jacket 136 through an outlet tube 216 at another location, as indicated by arrows 134, 135, respectively. The cooling fluid can be water supplied by a thermostatic circulator manufactured by Recirculating Chiller, Neslab Instruments, Waltham, Mass., or similar device. Any suitable thermostat 218, such as a thermocouple or other technology can be used to measure temperature of the exhaled breath flowing in the collection chamber 130 and to feed such measurements back to the microprocessor 192 or other suitable controller, as indicated schematically by link 220 (FIG. 10), to control the source 184 to produce more or less cooling as necessary to maintain the desired or prescribed temperature.

Upon entering the exhaust tube 132, as indicated by flow arrow 129, the exhaled breath flow, stripped of most, if not all, of the aerosol analytes 121, which cling to the collection surface 122, continues its flow through the exhaust tube 132, as indicated by flow arrow 198. One or more ports 200 near the top 202 of the exhaust tube 132 allow the exhaled breath to flow into an exhaust manifold chamber 204, as indicated by flow arrow 206. From the exhaust manifold chamber 204, the exhaled breath flows through an exhaust port fitting 208, as indicated by flow arrow 210, through the valve 164 and exhaust outlet conduit 178, as indicated by flow arrow 212, to the vacuum source 142. All of these exhaust components from the exhaust tube 132 to the exhaust port fitting 208 are sometimes referred to as an exhaust outlet.

Upon completion of a collection period, the exhaust tube 132, which is slidably sealed by a pair of O-rings 222, 224 or other appropriate seals, can be pulled longitudinally out of the collection chamber 130, as indicated by arrow 226 above the pull knob 228 at the top end 202 of the exhaust tube 132. Then, with the exhaust tube 132 pulled out of the collection chamber 130, the wiper 124 can be pushed by a rod 125 or other suitable device, either manually or with some machine actuator, spring, pneumatic or hydraulic actuator, etc., upwardly through the collection chamber 130 to wipe the analytes 121 off the collection surface 122. In addition to the analytes 121, there will be a significant amount of condensed water on the collection surface 122, which gets wiped along with the analytes off the surface 122 by the wiper 124 and will usually be adequate to dissolve the analytes 121 and retain them in solution. As the wiper 124 approaches the top end of the collection chamber 130, any suitable appliance or apparatus can be used to extract the condensed water and analytes from the collector 120 for further study, analysis, or other use. For example, a syringe or pipette (not shown) can be used to draw the solution containing the analytes out of the collection chamber 130 through the opening left by the removal exhaust tube 132, as will be understood by persons familiar with those kinds of instruments, or more sophisticated or automated equipment can be devised for this purpose.

The criteria for selecting particular physical dimensions and operating parameters for the collector 120 should preferably balance the efficiency and effectiveness of the aerosol collection against the dilution caused by the condensed water. It may be preferable, but not essential, that the temperature differential discussed above be increased only to the extent that further increase no longer increases the amount of detectable analytes in the collected specimen. Further, the collection chamber 130, while shown to be cylindrical, can also be conical, spherical, or any other shape, but is preferably a figure of revolution. Also, all of the control features described above, including, but not limited to, those described for the collector 10 of FIGS. 1-9 can be used in this collector embodiment 120, as will be understood by persons skilled in the art, once they understand the principles of this invention.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Collection and Analysis

The invention uniquely couples a high-efficiency, essentially lossless sample collector operationally to a rapid high-sensitivity analyzer to provide sample-to-answer analysis directly from the sample source, such as exhaled breath from a medical patient or a suspected substance abuser.

Analytical chemistry often uses molecular separation methods, including numerous variations of chromatography, mass spectrometry, and electrophoresis (itself a variant of chromatographic methods). Commercial examples exist for each of these major modalities implemented at “chip” scale that can provide extremely short analysis times (from milliseconds to minutes) and extremely small analyte quantities (e.g., picomoles of analyte). Ion mobility spectrometry (IMS) and capillary electrophoresis (CE) provide two practical examples.

These analytical methods readily provide very broad-spectrum capability in terms of the types and sizes of molecular analytes to be detected, from inorganic ions to large DNA fragments (as in commercial CE DNA sequencing systems that use chip-scale disposable analyzers).

In CE, for example, electrokinetic separations are carried out in narrow-lumen (˜10-50 μm) capillaries at high voltages (5-30 kV for macro-scale instruments using 0.5-1 meter capillaries), and producing plug flow, thus achieving high efficiency (N>100,000 theoretical plates), resolution, and mass sensitivity (sub-attomolar for some analytes). Main CE characteristics include versatility of application, use of different separation modes and matrices with different selectivity, extremely small sample volume, negligible running costs, low-cost capillary chips, possibility of interfacing with different “hyphenated” detection systems (sequential analyzer stages), and the ruggedness and simplicity of the instrumentation. Several types of high-sensitivity detector can be integrated onto the capillary tubing, including electrochemical detectors and laser-induced fluorescence, without adding significant cost to a disposable analyzer chip.

IMS has characteristics that closely resemble those for CE, using a drift tube filled with neutral gas near atmospheric pressure instead of liquid phase separation as in CE or a high vacuum as in mass spectrometry. Both CE and IMS require release of the analyte molecules from the original sample matrix. With CE the electrophoresis sample buffer suffices. With IMS, solvent elution provides a direct analyte solution for electrospray injection into the spectrometer. Both methods measure and identify mobile components by band intensity (AUC, area under the curve of a discrete peak) vs. time. Both methods require optimization of analysis conditions to achieve best sensitivity and specificity for a targeted analyte or group of analytes.

In its preferred embodiment, the invention takes advantage of the multi-stage electrical fields in the collector and analyzer subsystems to directly couple the collector outflow to the CE input. This continuous coupling eliminates the need for mechanical transfers between the collection subsystem and the analyzer subsystem. The transition from collection to analysis occurs by terminating the collector's electrical field and activating the analyzer's electrical field.

Most drugs are small-molecule organic compounds. By virtue of being transported within the body and into cells to reach their sites of action, they are designed for aqueous environments. Drugs that cross the blood-brain barrier have additional lipophilic properties that enable transport and diffusion within the central nervous system. Analytical assay design exploits a very large number of such variable properties.

As one example, delta-9-tetrahydrocannabinol (Δ-9-THC) is the most abundant psychoactive ingredient ingested or inhaled as smoke from marijuana plants, Cannabis spp. Secondary components, designated as cannabinoids, may also contribute to psychoactive effects, but Δ-9-THC provides the definitive target to identify recent marijuana use. Analytical principles used by the invention to measure Δ-9-THC can be changed as needed to detect other kinds of drugs, based on well-understood adaptations for different molecular targets.

Well-established analytical protocols are in use for Δ-9-THC laboratory detection and quantitation for forensic purposes. Numerous adaptations and innovations could conceivably become standard forensic methods. For example, one method is using CE for measuring multiple cannabinoids in a single sample and achieving two-log improvement in sensitivity relative to existing laboratory methods. The US Coast Guard, in another example, tested IMS for use in drug trafficking interdiction. High-performance liquid chromatography (HPLC) in many variants, gas chromatography of derivatized analytes, thin-layer chromatography, and highly sophisticated mass spectrometry in many variations and tandem modes have also been described.

The invention can also use programmable instrument control and analytical protocol libraries to execute device operations required by each particular application, such as real-time analysis for drugs of abuse or emergency toxicological diagnoses.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for determining the level of drug in a subject's system, said method comprising:
(i) collecting more than 95% of all aerosol particles from exhaled breath of a subject by having the subject exhale into a breath sample collecting apparatus and measuring the total volume of exhaled breath exhaled into the breath sample collecting apparatus;
(ii) determining the amount of a drug metabolite present in the collected aerosol particles;
(iii) normalizing the amount of the drug metabolite in the collected aerosol particles based on the volume of exhaled breath; and
(iv) determining the level of drug in the subject's system by using the normalized amount of the drug metabolite determined in said step (iii).

2. The method of claim 1, wherein said drug comprises cannabis.

3. The method of claim 2, wherein said drug metabolite comprises Δ-9-tetrahydrocannabionol; 11-hydrorxy-tetrahydrocannabionol; 11-nor-9-carboxy-tetrahydrocannabionol; Cannabinol, or a combination thereof.

4. The method of claim 1, wherein said drug comprises an opiate.

5. The method of claim 4, wherein said drug or drug metabolite comprises Acetyl-alpha-methylfentanyl (N-[-1-(1-methyl-2-phenethyl)-4-piperidinyl]-N-phenylacetamide); Acetylmethadol; Allylprodine; Alphacetylmethadol; Alphameprodine; Alphamethadol; Alpha-methylfentanyl; Alpha-methylthiofentanyl; Benzethidine; Betacetylmethadol; Beta-hydroxyfentanyl; Beta-hydroxy-3-methylfentanyl; Betameprodine; Betamethadol; Betaprodine; Clonitazene; Dextromoramide; Diethylthiambutene; Difenoxin; Diampromide; Dimenoxadol; Dimepheptanol; Dimethylthiambutene; Dioxaphetyl butyrate; Dipipanone; Ethylmethylthiambutene; Etonitazene; Etoxeridine; Furethidine; Hydroxypethidine; Ketobemidone; Levomoramide; Levophenacylmorphan; 3-Methylfentanyl; 3-Methylthiofentanyl; Morpheridine; MPPP (1-methyl-4-phenyl-4-propionoxypiperidine); Noracymethadol; Norlevophanol; Normethadone; Norpipanone; Para-fluorofentanyl; PEPAP (1-(-2-phenethyl)-4-phenyl-4-acetoxypiperidine); Phenadoxone; Phenampromide; Phenomorphan; Phenoperidine; Piritramide; Proheptazine; Properidine; Propiram; Racemoramide; Thiofentanyl (N-phenyl-N-[1-(2-thienyl)ethyl-4-piperidinyl]-propamide); Tilidine; Trimeperidine; Acetorphine; Acetyldihydrocodeine; Benzylmorphine; Codeine methylbromide; Codeine-N-Oxide; Cyprenorphine; Desomorphine; Dihyromorphine; Drotebanol; Etorphine; Heroin; Hydromorphinol; Methyldesorphine; Methyldihydromorphine; Morphine methylbromide; Morphine methylsulfonate; Morphine-N-Oxide; Myrophine; Nicocodeine; Nicomorphine; Normophine; Pholcodine; and Thebacon. Other opiates and opiate derivatives that can be tested using methods of the invention include, but are not limited to, Raw opium; Opium extracts; Opium fluid; Powdered opium; Granulated opium; Tincture of opium; Codeine; Dihydroetorphine; Ethylmorphine; Etorphine hydrochloride; Hydrocodone; Hydromorphone; Metopon; Morphine; Oxycodone; Oxymorphone; Thebaine; Alfentanil; Alphaprodine; Anileridine; Bezitramide; Bulk dextropropoxyphene; Carfentanil; Dihydrocodeine; Fentanyl; Isomethadone; Levo-alphacetylmethadol (LAAM); Levomethorphan; Levorphanol; Metazocine; Methadone; Methadone-intermediate (4-cyano-2-demethylamino-4,4-diphenyl butane); Moramide-intermediate (2-methyl-3-morpholino-1,1-diphenylpropane-carboxylic acid); Pethidine (meperidine); Pethidine-intermediate-A (4-cyano-1-methyl-4-phenylpiperidine); Pethidine-intermediate-B (ethyl-4-phenylpiperidine-4-carboxylate); Pethidine-intermediate-C (1-methyl-4-phenylpiperidine-4-carboxylic acid); Phenazocine; Piminodine; Racemethorphan; Racemorphan; Remifentanil; Sufentanil, or a mixture thereof.

6. The method of claim 1, wherein said drug or drug metabolite comprises Amphetamine; Methamphetamine; Amphetamine-d₅; THC; Morphine; 6-acetymorphine; Cocaine; Benzoylecgonine; Diazepam; Oxazepam; Buprenorphine; Methylphenidate/ritalinic acid; Tramadol, or a combination thereof.

7. The method of claim 1, wherein said drug or drug metabolite comprises Alpha-ethyltryptamine (etryptamin, Monase, AET, a-AT); 4-Bromo-2,5-dimethoxy-amphetamine (4-bromo-2,5-DMA; 4-bromo-2,5-dimethoxy-a-methylphenethylamine); 4-Bromo-2,5-dimethoxy-phenethylamine (alpha-desmethyl DOB; 2C-B, Nexus); 2,5-Demethoxy-amphetamine (2,5-dimethoxy-a-methylphenethylamine; 2,5-DMA); 2,5-Dimethoxy-4-ethyl-amphetamine (DOET); 4-Methoxyamphetamine (4-methoxy-a-methyl-phenethylamine; PMA); 5-Methoxy-3,4-methylenedioxy-amphetamine; 4-Methyl-2,5-dimethoxy-amphetamine (4-methyl-2,5-dimethoxy-methylphenethylamine; DOM, STP); 3,4-Methylenedioxy-amphetamine (MDA); 3,4-Methylenedioxy-methamphetamine (MDMA); 3,4-Methylenedioxy-N-ethylamphetamine (N-ethyl MDA, MDE, MDEA); N-hyroxy-3,4-methylenedioxy-amphetamine (N-hydroxy MDA); 3,4,5-Trimethoxy-amphetamine; Bufotenine (3-(b-Dimethylaminoethyl)-5-hydroxyindole; 3-(2-dimethylaminoethyl) 5-indolol; N,N-dimethylserotonin; 5-hydroxy-N,N-dimethyltryptamine; mappine); Diethyltryptamine (DET); Dimethyltryptamine (DMT); Ibogaine (Tabermanthe iboga; 7-Ethyl-6,6-b, 7,8,9,10,12,13-octahydro-2-methoxy-6,9-methano-5H-pyrido (1′,2′:1,2) azepino (5,4-b) indole); Lysergic acid diethylamide (LSD); Marihuana; Mescaline; Parahexyl (Synhexyl; 3-Hexyl-1-hydroxy-7,8,9,10-tetrahydro-6,6,9-trimethyl-6H-dibenzo(b,d)pyran); Peyote (all parts of the plant Lophosphora williamsii Lemaire); N-ethyl-3-piperidyl benzilate; N-methyl-3-piperidyl-benzilate; Psilocybin; Psilocyn; Tetrahydrocannabinols; Ethylamine analog of phencyclidine (PCE; cyclohexamine; N-ethyl-1-phenylcyclohexylamine); Pyrrolidine analog of phencyclidine (PCPy; PHP; 1-(1-phenylcyclohexyl)-pyrrolidine); Thiophene analog of phencyclidine (TPCP; TCP; 1-(1-(2-thienyl)-cyclohexyl)-piperidine); 1-(1-(2-Thienyl)cyclohexyl)pyrrolidine (TCPy); Gamma-hydroxybutyrate (GHB); Mecloqualone; Methaqualone; Aminorex (aminoxaphen; 2-amino-5-phenyl-2-oxazoline; 4,5-dihydro-5-phenyl-2-oxazolamine); Cathinone (norephedrone; 2-amino-1-phenyl-1-propanone; alpha-aminopropiophenone; 2-aminopropiophenone); Fenethylline; Methcathinone (ephedrine; methylcathinone; 2-(methylamino)-propiophenone; alpha-(methylamino)-propiophenone; monomethylpropion); (+/−)cis-4-methylaminorex; N-ethylamphetamine; N,N-dimethylamphetamine (N,N-alpha-trimethyl-benzeneethanamine; N,N-alpha-trimethylphenethylamine); N-(1-benzyl-4-piperidyl)-N-phenylpropanamide (benzylfentanyl); N-(1-(2-thienyl)methyl-4-piperidyl)-N-phenylpropanamide (thenylfentanyl); Amphetamine; Methamphetamine; Phenmetrazine; Methylphenidate; Amobarbital; Glutethimide; Pentobarbital; Phencyclidine (PCP); Secobarbital; Nabilone; Phenylacetone (P2P, phenyl-2-propanone, benzylmethyl ketone); 1-Phenylcyclohexylamine; 1-Piperidinocyclohexanecarbontrile (PCC), or a mixture thereof.

8. The method of claim 1, wherein said step (iv) of determining the level of drug in the subject's system comprises comparing the normalized amount of the drug metabolite with a control.

9. The method of claim 1, wherein said step (ii) of determining the amount of a drug metabolite present in the collected aerosol particles comprises:
diluting said collected aerosol particles with a solvent to produce a sample solution; and
determining the amount of drug metabolite in the sample solution.

10. The method of claim 9, wherein the amount of drug metabolite in the sample solution is determined by a chromatography, mass spectrometer, nuclear magnetic resonance, infrared spectrometer, ultra violet/visible light (UV/VIS) spectrometer, capillary electrophoresis, or a combination thereof.

11. The method of claim 1, wherein said breath sample collecting apparatus comprises:
(a) a flow meter for measuring the volume of exhaled breath collected from the subject;
(b) an aerosol collection chamber with a collection surface charged with an electrostatic voltage for collecting aerosol particles from exhaled breath, wherein the aerosol particles are ionized after being exhaled;
(c) a conduit for channeling the exhaled breath from the subject to the aerosol collection chamber;
(d) an ionizer system in the conduit for ionizing the aerosol particles in the exhaled breath, an extractor system to remove the aerosol particles from the collection surface for analysis; and
(e) a pre-collection filter, wherein the pre-collection filter is an ionizing filter connected in fluid-flow relation to the conduit, and the pre-collection filter is positioned in close enough proximity to the aerosol collection chamber to filter ambient aerosols and prevent ambient aerosols from being inhaled by the test subject.

12. A method for determining the presence of drug in a subject's system, said method comprising:
(i) collecting more than 95% of all aerosol particles from exhaled breath of a subject by having the subject exhale into a breath sample collecting apparatus and measuring the total volume of exhaled breath exhaled into the breath sample collecting apparatus;
(ii) determining the amount of a drug metabolite present in the collected aerosol particles;
(iii) normalizing the amount of the drug metabolite in the collected aerosol particles based on the volume of exhaled breath; and
(iv) determining the presence of drug in the subject's system by using the normalized amount of the drug metabolite determined in said step (iii).

13. The method of claim 12, wherein said step (iv) of determining the presence of drug in the subject's system comprises comparing the normalized amount of drug metabolite with a control value.
